SEQUENCE LISTING (39 sequences)

SEQ ID NO: 1 - DNA, 62 bp, Artificial Sequence
  Template UEI-DraIII
gagcggataacaatttcacacaggcacggggtgtgagatcacagatcggaagagcgtcgtgt

SEQ ID NO: 2 - DNA, 24 bp, Artificial Sequence
  UEI-Fwd
gagcggataacaatttcacacagg

SEQ ID NO: 3 - DNA, 19 bp, Artificial Sequence
  UEI-Rev
acacgacgctcttccgatc

SEQ ID NO: 4 - DNA, 142 bp, Artificial Sequence
  Template Calibrator-N15-AlwNI
  misc_feature (86)..(110): n is a, c, g, or t
tcgtcggcagcgtcagatgtgtataagagacagggaagacgtagcaagggagtagccaatgtgaattgagagccttaagctgtatnnnnnnnnnnnnnnnnnnnnnnnnncaggggctggtcgtgactgggaaaaccctggc

SEQ ID NO: 5 - DNA, 23 bp, Artificial Sequence
  Calib-Rev
gccagggttttcccagtcacgac

SEQ ID NO: 6 - DNA, 19 bp, Artificial Sequence
  Illu-2-Fwd
tcgtcggcagcgtcagatg

SEQ ID NO: 7 - DNA, 67 bp, Artificial Sequence
  Template UEI-N15-DraIII*
  misc_feature (34)..(48): n is a, c, g, or t
gagcggataacaatttcacacaggcacggggtgnnnnnnnnnnnnnnngatcggaagagcgtcgtgt

SEQ ID NO: 8 - DNA, 149 bp, Artificial Sequence
  Template Calibrator-N15-AlwNI-lg
  misc_feature (103)..(117): n is a, c, g, or t
tcgtcggcagcgtcagatgtgtataagagacagggaagacgtagcaagggagtagccaatgtgaattgagagccttaagctgtatcaggaccagagagatgannnnnnnnnnnnnnncaggggctggtcgtgactgggaaaaccctggc

SEQ ID NO: 9 - DNA, 53 bp, Artificial Sequence
  Illu1 rev UEI
gtctcgtgggctcggagatgtgtataagagacagacacgacgctcttccgatc

SEQ ID NO: 10 - DNA, 19 bp, Artificial Sequence
  Illu1
gtctcgtgggctcggagat

SEQ ID NO: 11 - DNA, 90 bp, Artificial Sequence
  Template-AlwNI-N15-Calib-DraIII
  misc_feature (44)..(58): n is a, c, g, or t
ggaagacgtagcaagggagtagccaatgtgaattcacacagtgnnnnnnnnnnnnnnncaggggctggtcgtgactgggaaaaccctggc

SEQ ID NO: 12 - DNA, 24 bp, Artificial Sequence
  Calib-Fwd
ggaagacgtagcaagggagtagcc

SEQ ID NO: 13 - DNA, 80 bp, Artificial Sequence
  RTmimicks
  misc_feature (36)..(43): n is a, c, g, or t
ctgcgcggatcccggaagcgaggccagctggctgcnnnnnnnnttggctgtctcttatacacatctgacgctgccgacga

SEQ ID NO: 14 - DNA, 38 bp, Artificial Sequence
  antiSBACA-AlwN1
gcagccagctggcctcgcttccgggatccgcgcagaca

SEQ ID NO: 15 - DNA, 101 bp, Artificial Sequence
  Template UEI-N15-DraIII-Illu1
  misc_feature (34)..(48): n is a, c, g, or t
gagcggataacaatttcacacaggcacggggtgnnnnnnnnnnnnnnngatcggaagagcgtcgtgtctgtctcttatacacatctccgagcccacgagac

SEQ ID NO: 16 - RNA, 539 nt, Staphylococcus aureus
auuaauacgacucacuauaccuagaucacagagaugugauggaaaauaguugaugaguuguuuaauuuuaagaauuuuuaucuuaauuaaggaaggagugauuucaauggcacaagauaucauuucaacaaucggugacuuaguaaaauggauuaucgacacagugaacaaauucacuaaaaaauaagaugaauaauuaauuacuuucauuguaaauuuguuaucuacguauaguacuaaaaguaugaguuauuaagccaucccaacuuaauaaccauguaaaauuagcaagugaguaacauuugcuaguagaguuaguuuccuuggacucagugcuauguauuuuucuuaauuaucauuacagauaauuauuucuagcauguaagcuaucguaaacaacaucgauuuaucauuauuugauaaauaaaauuuuuuucauaauuaauaacauccccaaaaauagauugaaaaaauaacuguaaaacauucccuuaauaauaaguauggucgugagccccucccaagcucgcggccuuuug

SEQ ID NO: 17 - skipped

SEQ ID NO: 18 - DNA, 55 bp, Artificial Sequence
  RT-UMI-RNAIII*
  misc_feature (36)..(41): n is a, c, g, or t
ctgcgcggatcccggaagcgaggccagctggctgcnnnnnnttgggaggggctca

SEQ ID NO: 19 - skipped

SEQ ID NO: 20 - DNA, 35 bp, Artificial Sequence
  SB-alone
ctgcgcggatcccggaagcgaggccagctggctgc

SEQ ID NO: 21 - DNA, 63 bp, Artificial Sequence
  RNAIII-Fwd
tcgtcggcagcgtcagatgtgtataagagacagataactgtaaaacattcccttaataataag

SEQ ID NO: 22 - RNA, 867 nt, Artificial Sequence
  Gfp mRNA
augaccagcuacccauacgauguuccagauuacgcuaucgaaggccgcggccgccaucaucaucaucaucaugauaucgguaccaguaaaggagaagaacuuuucacuggaguugucccaauucuuguugaauuagauggugauguuaaugggcacaaauuuucugucaguggagagggugaaggugaugcaacauacggaaaacuuacccuuaaauuuauuugcacuacuggaaaacuaccuguuccauggccaacacuugucacuacuuucgcguauggucuucaaugcuuugcgagauacccagaucauaugaaacagcaugacuuuuucaagagugccaugcccgaagguuauguacaggaaagaacuauauuuuucaaagaugacgggaacuacaagacacgugcugaagucaaguuugaaggugauacccuuguuaauagaaucgaguuaaaagguauugauuuuaaagaagauggaaacauucuuggacacaaauuggaauacaacuauaacucacacaauguauacaucauggcagacaaacaaaagaauggaaucaaaguuaacuucaaaauuagacacaacauugaagauggaagcguucaacuagcagaccauuaucaacaaaauacuccaauuggcgauggcccuguccuuuuaccagacaaccauuaccuguccacacaaucugcccuuucgaaagaucccaacgaaaagagagaccacaugguccuucuugaguuuguaacagcugcugggauuacacauggcauggaugaacuauacaaagagaauucagagcucggauccacucgagaugcauuagaacaaaaauuauuaucagaagaagauuuaaauuaa

SEQ ID NO: 23 - DNA, 69 bp, Artificial Sequence
  gfp-mut2-RT-UMI-AlwNI*
  misc_feature (36)..(43): n is a, c, g, or t
ctgcgcggatcccggaagcgaggccagctggctgcnnnnnnnncaaataaatttaagggtaagttttcc

SEQ ID NO: 24 - DNA, 26 bp, Artificial Sequence
  Gfp-Fwd-25nt
tgaattagatggtgatgttaatgggc

SEQ ID NO: 25 - DNA, 70 bp, Artificial Sequence
  Template-UEI-DraIII-N2x5
  misc_feature (34)..(35): n is a, c, g, or t
  misc_feature (38)..(39): n is a, c, g, or t
  misc_feature (42)..(43): n is a, c, g, or t
  misc_feature (46)..(47): n is a, c, g, or t
  misc_feature (50)..(51): n is a, c, g, or t
gagcggataacaatttcacacaggcacggggtgnnacnngannctnngcnngatcggaagagcgtcgtgt

SEQ ID NO: 26 - DNA, 76 bp, Artificial Sequence
  Template-UEI-DraIII-N4443
  misc_feature (34)..(37): n is a, c, g, or t
  misc_feature (41)..(44): n is a, c, g, or t
  misc_feature (48)..(51): n is a, c, g, or t
  misc_feature (55)..(57): n is a, c, g, or t
gagcggataacaatttcacacaggcacggggtgnnnnacannnngagnnnntctnnngatcggaagagcgtcgtgt

SEQ ID NO: 27 - DNA, 85 bp, Artificial Sequence
  Template-Calibrator-AlwNI
ggaagacgtagcaagggagtagccaatgtgaattcacacagtgtctacaagtacaggggctggtcgtgactgggaaaaccctggc

SEQ ID NO: 28 - DNA, 93 bp, Artificial Sequence
  Template-Calibrator-AlwNI-N2x5
  misc_feature (44)..(45): n is a, c, g, or t
  misc_feature (48)..(49): n is a, c, g, or t
  misc_feature (52)..(53): n is a, c, g, or t
  misc_feature (56)..(57): n is a, c, g, or t
  misc_feature (60)..(61): n is a, c, g, or t
ggaagacgtagcaagggagtagccaatgtgaattcacacagtgnntcnntanncannagnncaggggctggtcgtgactgggaaaaccctggc

SEQ ID NO: 29 - DNA, 99 bp, Artificial Sequence
  Template-Calibrator-AlwNI-N4443
  misc_feature (44)..(47): n is a, c, g, or t
  misc_feature (51)..(54): n is a, c, g, or t
  misc_feature (58)..(61): n is a, c, g, or t
  misc_feature (65)..(67): n is a, c, g, or t
ggaagacgtagcaagggagtagccaatgtgaattcacacagtgnnnntctnnnnacannnngagnnncaggggctggtcgtgactgggaaaaccctggc

SEQ ID NO: 30 - DNA, 90 bp, Artificial Sequence; identical to SEQ ID NO: 11
  Template-Calibrator-AlwNI-N15
  misc_feature (44)..(58): n is a, c, g, or t

SEQ ID NO: 31 - DNA, 100 bp, Artificial Sequence
  Tag-NaBAb-AlwNI
  misc_feature (32)..(39): n is a, c, g, or t
cacacaggaaacagctatgacccagtgtctgnnnnnnnntgtggacgctgtctcttatacacatctgacgctgccgacgacgtcgtgactgggaaaaccc

SEQ ID NO: 32 - DNA, 20 bp, Artificial Sequence
  BG-M13-Fwd**
gggttttcccagtcacgacg

SEQ ID NO: 33 - DNA, 22 bp, Artificial Sequence
  Alexa488-M13-Rev
cacacaggaaacagctatgacc

SEQ ID NO: 34 - DNA, 62 bp, Artificial Sequence
  Example of UEI sequence
  misc_feature (34)..(43): n is a, c, g, or t
gagcggataacaatttcacacaggcacggggtgnnnnnnnnnngatcggaagagcgtcgtgt

SEQ ID NO: 35 - DNA, 62 bp, Artificial Sequence
  example of UEI sequence
  misc_feature (20)..(29): n is a, c, g, or t
acacgacgctcttccgatcnnnnnnnnnncaccccgtgcctgtgtgaaattgttatccgctc

SEQ ID NO: 36 - DNA, 83 bp, Artificial Sequence
  Template UEI-N15-DraIII-Illu2
  misc_feature (34)..(48): n is a, c, g, or t
gagcggataacaatttcacacaggcacggggtgnnnnnnnnnnnnnnnaactgtctcttatacacatctgacgctgccgacga

SEQ ID NO: 37 - DNA, 66 bp, Artificial Sequence
  RNAIII-Fw-89-Lg
gtctcgtgggctcggagatgtgtataagagacagtcataattaataacatccccaaaaatagattg

SEQ ID NO: 38 - DNA, 38 bp, Artificial Sequence; identical to SEQ ID NO: 14
  primer used to reverse transcribe gfp mRNA (sequence of Figure 22)

SEQ ID NO: 39 - DNA, 69 bp, Artificial Sequence; identical to SEQ ID NO: 23
  primer used to reverse transcribe gfp mRNA (sequence of Figure 22)
  misc_feature (36)..(43): n is a, c, g, or t

(19) United States
(12) Patent Application Publication
(10) Pub. No.: US 2021/0214791 A1
(43) Pub. Date: Jul. 15, 2021

(54) ABSOLUTE QUANTIFICATION OF TARGET MOLECULES AT SINGLE-ENTITY RESOLUTION USING TANDEM BARCODING

(71) Applicants: UNIVERSITE DE STRASBOURG, STRASBOURG (FR); CENTRE NATIONAL DE LA RECHERCHE SCIENTIFIQUE, PARIS (FR)

(72) Inventors: MICHAËL RYCKELYNCK, STRASBOURG (FR); STÉPHANIE BAUDREY, LINGOLSHEIM (FR); ROGER CUBI PIQUE, STRASBOURG (FR)

(21) Appl. No.: 17/057,131

(22) PCT Filed: May 21, 2019
(86) PCT No.: PCT/EP2019/063156

§ 371 Date: Nov. 20, 2020

(30) Foreign Application Priority Data
May 22, 2018 (EP) 18305629.0

(51) Int. Cl. C12Q 1/6876; C12Q 1/6806

(52) U.S. Cl. CPC C12Q 1/6876; C12Q 1/6806

(57) ABSTRACT

The present invention relates to a new method of labelling any target molecules from a plurality of entities, preferably in high throughput regimes, i.e. allowing the analysis of several thousands of entities per run, while preserving the integrity of the single-entity information. This method is based on a tandem molecular barcoding in which all molecular targets are labelled with a first unique barcode which is different for each molecular target from an entity, and with a tag sequence coding the entity from which the molecular target originates. Once this tandem barcoding is performed, the absolute quantification of all molecular targets with a single-entity resolution may be carried out in a single run of next-generation sequencing. The present invention also relates to a method of quantifying one or several molecular targets from a plurality of entities with single-entity resolution, as well as a kit and the use of such kit to label a plurality of molecular targets from a plurality of entities according to the method of the invention.

### FIELD OF THE INVENTION

The present invention relates to methods and systems for labelling nucleic acids and other biological molecules from entities, e.g. cells, within emulsion droplets in high throughput regimes while preserving the integrity of the single-entity information.

### BACKGROUND OF THE INVENTION

Single cell analytics are gaining popularity due to the insight that taking into account the heterogeneity of a population of cell may be of capital interest to understand the function and behaviour of diverse biological systems. Individual cells of a population of clonal unicellular organisms or of multicellular organisms, even among cells of the same tissue or cell-type, often exhibit heterogeneous gene regulation or protein expression patterns. It is thus increasingly recognized that traditional analytical approaches that analyse large populations of cells yield ensemble views that, although informative, only reflect the dominant biological mechanism and fail to identify cell-to-cell variations.

RNA levels are considered a useful marker of phenotypic heterogeneity and, as a consequence, considerable efforts were done to analyse RNA content in single cells. Probe-dependent methods including fluorescence in situ hybridization (FISH) or reporter fusions to fluorescent proteins, was replaced with the probe-independent RNA-seq technique in which cellular RNA molecules are converted into cDNA and subsequently sequenced in parallel using next-generation sequencing technology. Single-cell RNA-seq requires the isolation of individual cells, the conversion of cellular RNA into cDNA and the massively parallel sequencing of cDNA libraries.

The rapid expansion of microfluidic devices has resulted in the development of valve-based microfluidic chips wherein cells are isolated in nano-liter reaction chambers (Streets et al., 2014). However, this approach remains limited not only by the cost but also because the number of single cells that can be currently processed with said chips remains at less than one hundred per run. Alternatively, microfluidic droplets also provide a compartment in which cells can be isolated. Typically, droplets of one phase are generated in another, immiscible phase by exploiting capillary instabilities in a microfluidic two-phase flow. The addition of a surfactant to either or both of the phases stabilizes the droplets against coalescence and allows them to function as discrete microreactors.

Furthermore, as single-cell RNA-seq analysis may require the profiling of several thousands if not millions of representative individual cells, barcoding strategies have been developed to reduce sequencing costs and increase throughput. Using unique cellular identifiers, it has made possible to pool up a multitude of cells for simultaneous sequencing since each read could subsequently be assigned to its original cell through the unique cellular barcode (Islam et al., 2012).

The main technical challenge when combining barcoding strategy and compartmentalization of cells into droplets is to ensure that each droplet carries a different barcode and thus that the integrity of the single cell information is preserved.

Several methods have been described to address this problem. Each cell may be co-encapsulated with a distinctly barcoded particle, such as bead (Macosko et al. 2015) or hydrogel microsphere (Klein et al., 2015), in a nano-liter scale droplet. Each of these particles contains more than 10⁸ individual primers that share the same “cell barcode”. In such a method, in order to ensure that a droplet comprises only one particle, the number of droplets created greatly exceeds the number of particles or cells injected, so that a droplet will generally contain zero or one cell and zero or one particle (Macosko et al. 2015; Klein et al., 2015).

Alternatively, a barcode-library emulsion may be produced using a microfluidic device consisting of 96 drop-makers creating millions of drops containing a high concentration of a single one of the 96 barcodes (Rotem et al. 2015). Each cell-bearing drop is then paired and fused with one barcode-drop. However, to ensure that each cell-bearing drop is fused with at most one barcode drop, only half of the cell-bearing drops actually fuse with a barcode drop. Furthermore, cases where two cell-bearing drops fuse with a single barcode drop or where two barcode drops fuse with a single cell-bearing drop, introduce errors in the resultant labelling and are a potential source of noise (Rotem et al. 2015).

Thus, whatever the method used to ensure that each droplet carries a different barcode, this results in a limited droplet occupancy, a reduced useful fraction of droplets, and thus a reduced throughput.

Furthermore, even if RNA levels have been recognized as useful marker for phenotypic heterogeneity, current methods provide limited information since levels of protein or other biological molecules cannot be assessed with the same system. Indeed, to date, quantification of the protein expression at the single cell level, which is critical for complete characterization of the phenotypic states, is generally based on fluorescence imaging methods.

Consequently, there is a great need for new methods and devices allowing high-throughput quantification of RNA, proteins and other biological molecules of interest at the single entity level.

### SUMMARY OF THE INVENTION

The present invention provides a new method of labelling any target molecules from a plurality of entities in high throughput regimes while preserving the integrity of the single-entity information.

Accordingly, in a first aspect, the present invention relates to a method of labelling a plurality of molecular targets from a plurality of entities while preserving the integrity of the single-entity information, said method comprising
providing a first set of emulsion droplets comprising droplets containing molecular targets, wherein each of these droplets comprises a plurality of molecular targets originating from no more than one entity;
providing a second set of emulsion droplets comprising droplets containing probes,

wherein each probe comprises a capture moiety capable of specific binding or ligation to a molecular target contained in droplets of the first set or to an adaptor linked to said molecular target, and a DNA moiety comprising an identification sequence,

wherein each identification sequence comprises a molecular identification (UMI) barcode, an entity identification (UEI) barcode and a calibrator (UEI-calibrator) barcode,

wherein each droplets of the second set comprises one or several UEI barcodes and one or several UEI-calibrator barcodes, the combination of UEI barcodes and UEI-calibrator barcodes being different for each droplet of the second set, and

wherein each identification sequence contained in a droplet of the second set comprises a UMI barcode which is different from the other identification sequences contained in the same droplet,
fusing droplets of the first set with droplets of the second set wherein a droplet of the first set is fused with no more than one droplet of the second set; and
labelling each molecular target with an identification sequence.

The method may further comprise encapsulating a plurality of entities within emulsion droplets, each droplet containing no more than one entity, and optionally lysing said entities within the droplets to release molecular targets, thereby obtaining the first set of emulsion droplets.

The method may further comprise

encapsulating a plurality of entity identification (UEI) sequences, a plurality of calibrator (UEI-calibrator) sequences and a plurality of molecular identification (UMI) molecules with an amplification reaction mixture within emulsion droplets

wherein each droplet comprising one or several UEI sequences, one or several UEI-calibrator sequences and a plurality of UMI molecules, the combination of UEI sequences and UEI-calibrator sequences being different for each droplet and each droplet comprising a plurality of UMI molecules,

wherein each UEI sequence comprises a UEI barcode and one or two overhang producing restriction sites,

each UEI-calibrator sequence comprises a UEI-calibrator barcode and one or two overhang producing restriction sites,

each UMI molecule comprises a capture moiety capable of specific binding or ligation to a molecular target or to an adaptor linked to said molecular target, and a DNA moiety comprising (i) a region proximal to the capture moiety and comprising a UMI barcode and (ii) a region distal from the capture moiety and comprising an overhang or an overhang producing restriction site, and

each UMI molecule comprises a UMI barcode which is different from the other UMI molecules contained in the same droplet;

amplifying UEI sequences and UEI-calibrator sequences within droplets; and

assembling UEI-calibrator barcodes, UEI barcodes and UMI molecules through restriction enzyme digestion and ligation of compatible overhangs,

thereby obtaining the second set of emulsion droplets.

Preferably, (a) UEI sequences and UEI-calibrator sequences are assembled through restriction enzyme digestion and ligation of compatible overhangs before amplification, and then (b) UMI molecules and amplification products are assembled through restriction enzyme digestion and ligation of compatible overhangs.

In some embodiments, at least some of molecular targets are nucleic acids and at least some probes comprise a capture moiety which is a single stranded DNA region which drives the specific recognition of a nucleic acid molecular target through conventional Watson-Crick base-pairing interactions.

Said nucleic acid molecular targets may be labelled using said probes as priming sites for a DNA polymerase synthetizing complementary strands of molecular targets.

In particular embodiments, at least some of molecular targets are RNA molecules and the DNA polymerase is a reverse transcriptase.

In some embodiments, at least some probes comprise a capture moiety which is

(i) a binding moiety that specifically binds to a molecular target and is directly bound to the DNA moiety,

(ii) a chimeric protein comprising a first domain that specifically binds to a molecular target and a second domain that binds to the DNA moiety, or

(iii) a binding moiety that binds specifically to a molecular target and a protein bridge, said protein bridge comprising a first domain that binds to the binding moiety and a second domain that binds to the DNA moiety.

Preferably, (i) the binding moiety or the first domain of the chimeric protein is selected from the group consisting of an antibody, a ligand of a ligand/anti-ligand couple, a peptide aptamer, a nucleic acid aptamer, a protein tag, or a chemical probe (e.g. suicide substrate, activity-based probes ABP) reacting specifically with a molecular target or a class of molecular targets, preferably is an antibody, (ii) the first domain of the protein bridge is an immunoglobulin-binding bacterial protein, preferably is domains A to E of protein A, and/or (iii) the second domain of the protein bridge or the chimeric protein is selected from the group consisting of SNAP-tag, CLIP-tag or Halo-Tag, preferably is a SNAP-tag.

Preferably, at least some probes comprise a capture moiety comprising an antibody moiety specific to a molecular target and a protein bridge, said protein bridge comprising a first domain that binds to a Fc region of the antibody moiety and a second domain that binds to the DNA moiety, preferably a SNAP-tag.

Preferably, at least one step of the method is implemented using a microfluidic system. In particular, a microfluidic system may be used to generate the first set of emulsion droplets, and/or a microfluidic system may be used to generate the second set of emulsion droplets, and/or a microfluidic system may be used to fuse droplets of the first set with droplets of the second set. More preferably the method is implemented using a microfluidic comprising
a first emulsion re-injection module or on-chip droplet generation module;
a second emulsion re-injection module or on-chip droplet generation module
a droplet-pairing module, and
optionally a module coupling droplet fusion to injection,

wherein emulsion re-injection modules and/or on-chip droplet generation modules are in fluid communication and upstream to the droplet-pairing module, the droplet-pairing module is in fluid communication and upstream to the module coupling droplet fusion to injection.

More preferably, all steps of the method of labelling of the invention is implemented using one or several microfluidic systems.

The present invention also relates to a method of quantifying one or several molecular targets from a plurality of entities with single-entity resolution, said method comprising

labelling said molecular targets according to the method of the invention;

capturing said labelled molecular targets,

amplifying sequences comprising UMI, UEI and UEI-calibrator barcodes,

sequencing amplified sequences.

In the methods of the invention, the entity may be a cell, a particle or an emulsion droplet, preferably an oil-in-water emulsion droplet exposing molecular targets on its outer surface.

The present invention further relates to a kit and the use of a kit to label a plurality of molecular targets from a plurality of entities according to the method of the invention, or to quantify one or several molecular targets from a plurality of entities with single-entity resolution according to the method of the invention, wherein the kit comprises
a microfluidic device and/or,
one or several probes as defined herein; and/or
one or several UMI molecules as defined herein; and/or
one or several UEI sequences as defined herein; and/or
one or several UEI-calibrator sequences as defined herein; and/or
one or several primers suitable to amplify UEI sequences and/or UEI-calibrator sequences as defined herein; and

optionally
an aqueous phase and/or an oil phase; and/or
a leaflet providing guidelines to use such a kit.

Preferably, the microfluidic device comprises
a first emulsion re-injection module or on-chip droplet generation module;
a second emulsion re-injection module or on-chip droplet generation module
a droplet-pairing module, and
optionally a module coupling droplet fusion to injection,

wherein emulsion re-injection modules and/or on-chip droplet generation modules are in fluid communication and upstream to the droplet-pairing module, the droplet-pairing module is in fluid communication and upstream to the module coupling droplet fusion to injection.

### BRIEF DESCRIPTION OF THE DRAWINGS

FIG. 1: Droplet-based microfluidics platforms for single cell molecular labeling. A. Single cell individualization and lysis. An aqueous stream containing the cells is combined with a stream of aqueous solution containing a lysis agent and optionally a double strand specific DNase. The emulsion is generated, collected and incubated to allow cell lysis and DNA degradation to occur. B. Droplet fusion. Droplets containing cell lysate are reinjected into a droplet fusion microfluidic chip and synchronized with on-chip generated droplets containing labeling mixture (reverse transcription mixture, probes, antibodies . . . ). Pairs of droplets are then fused when passing between a pair of electrodes at the fusion point (arrow).

FIG. 2: Exemplary embodiment of a DNA UMI molecule.

FIG. 3: Exemplary embodiment with a DNA UMI molecule comprising a capture moiety driving the specific recognition of a DNA adaptor linked to RNA molecular targets. The pre-adenylated adaptor (5′-App oligonucleotide) acts as a substrate for T4 ligase and is thus ligated to RNA molecules. The capture moiety then specifically hybridizes with the DNA adaptor.

FIG. 4: Exemplary embodiments with a DNA UMI molecule comprising a capture moiety which is a 5′ single stranded DNA region comprising 5′,5′-adenyl pyrophosphoryl moiety (App) onto its 5′-end. Such moiety acts as a substrate for T4 ligase and is thus ligated to RNA molecules.

FIG. 5: Exemplary embodiment of chimeric UMI molecule. A. Aptamer-based UMI molecule. This molecule is composed of a RNA or DNA aptamer specific of the target molecule and fused to a synthetic DNA labeling moiety. B. Chimeric UMI molecule comprising a capture moiety comprising a protein bridge (SNAP-Tag and protein A) and an antibody specific of the molecular target. C. Schematic organization of the capture moiety.

FIG. 6: Exemplary embodiment of UEI sequence. In this embodiment, the UEI sequence comprises a constant region, a unique restriction site, a UEI barcode and a sequencing primer annealing sequence.

FIG. 7: Exemplary embodiment of UEI-Calibrator sequence. The molecule is shown as a PCR-amplification product (double-stranded DNA). The UEI-Calibrator sequence comprises a sequencing primer annealing sequence, a spacer, a UEI-calibrator barcode, a unique restriction site and a constant region comprising a primer binding site allowing amplification of UEI-Calibrator sequence.

FIG. 8: Exemplary embodiment with UEI-calibrator sequences comprising two overhang producing restriction sites (RS), a first restriction site generating an overhang compatible with overhangs of digested UEI sequences comprising UEI barcodes and a second restriction site generating an overhang compatible with overhangs of UMI molecules. In this embodiment, the digestion/ligation step used to assemble the identification sequence leads to the formation of tripartite molecules comprising, from the capture moiety to the other extremity, UMI, UEI and UEI-calibrator barcodes.

FIG. 9: Co-flow droplet generator. The key dimensions of the microfluidic device are indicated. The depth of the channels was 10 μm.

FIG. 10: Droplet fluorescence analyzer. The key dimensions of the microfluidic device are indicated. The depth was 15 μm. Fluorescence measurement point is indicated by the open arrow.

FIG. 11: Fluorescence profile of orange-labelled droplets containing intact or lysed bacteria. Top panel: fluorescence profile of droplet containing intact bacteria. Each orange peak corresponds to a droplet. Green spikes observed into each orange peak corresponds to a fluorescent particle, therefore an intact bacterium. Bottom panel: fluorescence profile of droplet containing lyzed bacteria. Each orange peak corresponds to a droplet. Moreover, the presence of homogeneous green having the same width as the orange peak (e.g. the second peak from the left) indicates that the fluorescently-labelled nucleic acids have been released into the droplet, so that the bacterium has been lyzed.

FIG. 12. Bright-field and green fluorescence imaging of the water-in-oil droplets. The bacteria (green particles, arrows) encapsulated in the presence of CutSmart® buffer but in the absence of B-PER™ are shown on the left side whereas the bacteria encapsulated in the presence of B-PER™ are shown on the right side. Note that the lower number of fluorescent droplets after bacteria lysis is due to the more than a thousand-fold dilution of the fluorescence in the droplets following bacteria lysis, making these droplets difficult to distinguish from the background.

FIG. 13: Main steps in Unique Identifiers (UI) preparation (UI comprising UEI and UEI-calibrator barcodes).

FIG. 14: Analysis of PCR amplification and (co)-amplification of UEI-Calibrator sequences and UEI sequences. Left panel: analysis of the PCR amplification of the UEI sequences (lane 1), the UEI-Calibrator sequences (lane 2) or of both together (lane 3). Right panel: analysis of the PCR co-amplification of UEI-Calibrator sequences and UEIs in bulk (lane 4) and in droplets (lane 5). The position of the expected size for UEI-Calibrators and of the UEIs amplification products size are labelled respectively by an open and a closed arrow. The lane L corresponds to the low range ladder (SM1203, Fermentas). Both gels were 8% native polyacrylamide-TBE 1× gels.

FIG. 15: Droplet generator. The key dimensions of the microfluidic device are indicated and the channels were 40 μm deep.

FIG. 16. Droplet picoinjector. Key dimensions are indicated and the channels were 40 μm deep. Ground and positive electrodes are shown in light and dark gray respectively.

FIG. 17. Analysis of DNA labelling efficiency. Top panel: The proper formation of an UI following the recombination of a UEI-Calibrator-bearing DNA (black square) with an UEI-bearing DNA (dashed squares) at the level of a restriction site (open square) brings annealing sites of primer 6 and 10 on the same DNA allowing for qPCR to take place. Middle panel: The Ct values are given for experiments performed both in bulk and in emulsion at both UEI-Calibrator/UEI ratios. Moreover, labelling reactions were performed in the presence (+) or in the absence (−) of restriction/ligation enzymes. Finally, for each reaction the number of the corresponding lane on analysis gel is given. Bottom left panel: analysis of qPCR products on 8% native polyacrylamide-TBE 1×. The lane L corresponds to the low range ladder (SM1203, Fermentas). The position of the product of expected size is indicated by the black arrow. Bottom right panel: gel purification of indexed library. The library of indexed UIs was purified on a 1% native agarose gel in TBE. The lane L corresponds to the 1 kb ladder (SM1163, Fermentas). The position of the product of expected size is indicated by the black arrow. The white dotted line box shows the band recovered for sequencing.

FIG. 18. Bioinformatics algorithm used to analyze sequencing data.

FIG. 19: Barcodes distribution and signature occurrence in droplets. Left panel: Distribution of UEI-Calibrators and UEIs in droplets. The distribution of the number of different barcode sequences per droplet is shown for the UEI-Calibrator (gray dashed bars) as well as for UEIs (open bars). Right panel: upon UIs clustering in Signatures, the occurrence of signature in the sequence pool was determined.

FIG. 20. Analysis of UI formation at various barcode lambda values. Left panel: qPCR analysis of UI formation. The proper formation of an UI upon the recombination of a UEI-Calibrator-bearing DNA (black square) with an UEI-bearing DNA (dashed squares) at the level of a restriction site (open square) brings annealing sites of primer 6 and 10 on the same DNA allowing for qPCR to take place. Ct values are given for the different lambda (number of different UEI-Calibrators and UEIs per droplet) tested. Right panel: analysis of qPCR products on 8% native polyacrylamide-TBE 1×. The lane L corresponds to the low range ladder (SM1203, Fermentas). The position of the product of expected size is indicated by the black arrow.

FIG. 21. Distribution profile of UEI-Calibrators and UEIs in the droplets. Occurrence at values 1 and 2 were intentionally removed as they contained significant sequencing noise.

FIG. 22. Impact of RT primer labelling on its functionality. A. Scheme representation of the primer used to reverse transcribe gfp mRNA. B. Alternative strategies to generate UI-labelled cDNAs. Whereas in a Post-RT labelling strategy (Top) UI is appended to the cDNA after reverse transcription took place; in the Pre-RT labelling strategy (Bottom) the RT primer is labelled prior to being used for reverse transcription. The Ct value obtained by qPCR using primers allowing for quantifying the amount of cDNA-UI product generated are given under the schematic. C. Reverse transcription of gfp mRNA and UI attachment were tested in the presence or in the absence of reverse transcriptase (RT) and/or restriction/ligation enzymes (Enzymes). Reaction efficiency was then verified by qPCR (Ct values given in the table) and the identity of the PCR products controlled by gel electrophoresis on a 1% agarose gel-TBE 1×. The position of the product of expected size is indicated by the black arrow.

FIG. 23. Scheme representation of the primer used to reverse transcribe the RNA-III.

FIG. 24. 2 pL droplet generator. The key dimensions of the devices are indicated. The channels were 10 μm deep.

FIG. 25. Microfluidic droplet fuser. Key dimensions are indicated, and the channels were 15 μm deep. Ground and positive electrodes are shown in light and dark gray respectively.

FIG. 26. Analysis of reverse transcription products. Top: upon reverse transcription, the RT primer is extended and contain annealing site of primer 21. The generated cDNA contains both primer-binding sites (20 and 21) and can be detected by qPCR. Ct values are given in the table. Bottom: analysis of qPCR products on 8% native polyacrylamide-TBE 1×. Gels on the left, the middle and the right correspond respectively to the experiment started with 1000, 100 and 10 RNA per droplet. Lanes 1, 4 and 7 are the negative controls, lanes 2, 5 and 8 correspond to the experiment performed in bulk and lanes 3, 6 and 9 correspond to the reaction performed in droplets. The lanes L correspond to the low range ladder (SM1203, Fermentas). The position of the product of expected size is indicated by the black arrows whereas small parasitic side products are shown by the open arrows.

FIG. 27. Analysis of PCR products on 1% agarose gel-TBE. UI were initially prepared with random region-free (lane 1), N2x5 (lane 2), N4443 (lane 3) and N15 (lane 4) templates. The position of the product of expected size is indicated by the black arrow. The black vertical bar shows the short parasitic side products.

FIG. 28. Workflow of the preparation of NaBAb-DNA/IgG complex.

FIG. 29. Preparation of NaBAb-DNA/IgG complex. Left panel: Incubation of BG-labelled fluorescent DNA with (lane 2) and without (lane 1) NaBAb protein. L indicates a molecular weight ladder. Right panel: NaBAb protein was incubated alone (lane 1), with BG-labelled fluorescent DNA (lane 2) as well as an increasing concentration of IgG (62.5 μg/mL on lane 3, 112 μg/mL on lane 4 and 225 μg/mL on lane 5). Upon incubation, the reaction products were loaded on a native polyacrylamide gel and the position of DNA molecule was revealed by imaging gel fluorescence (emitted by the Alexa488 conjugated with the DNA) without further staining.

FIG. 30. UEI/UEI-calibrator attachment to NaBAb-DNA complex. Left panel: qPCR analysis of grafting to DNA-labelled protein. The proper formation of UEI/UEI-calibrator combination upon the recombination of a UEI-Calibrator-bearing DNA (black square) with an UEI-bearing DNA (dashed squares) at the level of a restriction site (open square) as well as the attachment of the combination, via a compatible restriction site at the extremity of a DNA covalently attached to a protein brings annealing sites of primer 6 and 10 on the same DNA allowing for qPCR to take place. Ct values are given in the table. Right panel: analysis of qPCR products on 1% agarose gel-TBE 1×. The lane L corresponds to the low range ladder (SM1203, Fermentas). The position of the product of expected size is indicated by the black arrow.

FIG. 31. Barcoding chip. Key dimensions are indicated. The channels were 40 μm deep. Positive (dark grey) and negative (light gray) are indicated.

FIG. 32. Analysis of labeled and RNA III-derived cDNAs by capillary electrophoresis and high-throughput sequencing. A. An aliquot of indexed and purified RNA III-derived DNA was analyzed on a Bioanalyzer platform (Agilent). B. Upon QC validation, labelled cDNAs were then loaded on a V3-150 MiSeq cartridge and analyzed on a MiSeq device. The quality of the preparation is witnessed by the high read number and the high-quality score. Moreover, among the non-Phix reads, more than 99.7% % (0.2% unvalidated vs 77.4% of tagged RNA III-derived cDNA) turned to be molecules of interest (i.e. RNA III-derived DNA labelled with barcodes).

### DETAILED DESCRIPTION OF THE INVENTION

The inventors conceived a new method of labelling any target molecules from a plurality of entities in high throughput regimes, i.e. allowing the analysis of several thousands of entities per run, while preserving the integrity of the single-entity information. This method is based on a tandem molecular barcoding in which all molecular targets (nucleic acids, proteins, . . . ) are labelled (i) with a first unique barcode (unique molecular identification barcode or UMI barcode) which is different for each molecular target from an entity, and (ii) with a tag sequence coding the entity from which the molecular target originates, i.e. a combination of a unique entity identification barcode or UEI barcode and of a UEI calibrator barcode, said combination being different for each entity but identical for all molecular targets originating from the same entity. Once this tandem barcoding is performed, the absolute quantification of all molecular targets with a single-entity resolution may be carried out in a single run of next-generation sequencing making this method highly sensitive and cost-effective.

In a first aspect, the present invention relates to a method of labelling a plurality of molecular targets from a plurality of entities, said method comprising
providing a first set of emulsion droplets comprising droplets containing molecular targets, wherein each of these droplets comprises a plurality of molecular targets originating from no more than one entity;
providing a second set of emulsion droplets comprising droplets containing probes,

wherein each probe comprises a capture moiety capable of specific binding or ligation to a molecular target contained in droplets of the first set or to an adaptor linked to said molecular target, and a DNA moiety comprising an identification sequence,

wherein each identification sequence comprises a molecular identification (UMI) barcode, an entity identification (UEI) barcode and a calibrator (UEI-calibrator) barcode,

wherein each droplets of the second set comprises one or several UEI barcodes and one or several UEI-calibrator barcodes, the combination of UEI barcodes and UEI-calibrator barcodes being different for each droplet of the second set, and

wherein each identification sequence contained in a droplet of the second set comprises a UMI barcode which is different from the other identification sequences contained in the same droplet,
fusing droplets of the first set with droplets of the second set wherein a droplet of the first set is fused with no more than one droplet of the second set; and
labelling each molecular target with an identification sequence.

The method of the invention may be used to label molecular targets from any type of entities.

As used herein, the term “entity” refers to any entity comprising or exposing on its surface, molecular targets as defined below. In particular, this term refers to a cell, or refers to a particle or an emulsion droplet, preferably an oil-in-water emulsion droplet, exposing molecular targets on its outer surface.

As used herein, the term “cell” refers to a prokaryotic cell or a eukaryotic cell such as animal, plant, fungal or algae cell. The population of cells to be processed may be homogenous, i.e. comprising only one cellular type, or may be heterogeneous, i.e. comprising several cellular types. In some embodiments, the population of cells is obtained from a tissue sample, preferably an animal tissue sample, more preferably from a pathological sample such as a tumor sample. In some other embodiments, the population of cells is a population of bacterial, fungal or algae cells, preferably of bacterial or fungal cells. This population may comprise bacteria, fungi or algae of the same species or bacteria, fungi or algae of different species.

The terms “particle” and “bead” are used herein interchangeably and refer to any solid support, preferably a spherical solid support, of 50 nm to 10 μm in size which is suitable to expose one or several molecular targets on its outer surface. In particular, these terms may refer to polymer beads (e.g. polyacrylamide, agarose, polystyrene), latex beads, magnetic beads or hydrogel beads. Methods for covalent or non-covalent binding of molecular targets such as nucleic acids or proteins, to beads are well known by the skilled person and various techniques are commercially available. In particular, this binding may be carried out through reactive groups on the surface of the particle. For example, nucleic acids may be attached to the surface by carbodiimide-mediated end-attachment of 5′-phosphate and 5′-NH2 modified nucleic acids to respectively amino and carboxyl beads. Proteins may also be covalently or non-covalently attached to beads via any suitable method such as using sulphate, amidine, carboxyl, carboxyl/sulphate or chloromethyl modified beads.

The term “entity” may also refer to an emulsion droplet, preferably an oil-in-water emulsion droplet, exposing molecular targets on its outer surface. Molecular targets may be covalently or non-covalently attached to the droplet through reactive groups exposed on the surface of the droplets such as nitrilotriacetate which can specifically interact with his-tagged proteins, or through any other functional moiety which is able to covalently or non-covalently interact with a molecular target of interest. The skilled person may use any known method to produce such emulsion droplets exposing molecular targets on its outer surface, in particular methods described in international patent application WO 2017/174610.

The method of the invention allows labelling molecular targets from a high number of entities in a single run. Thus, as used herein, the term “plurality of entities” refers to at least 1,000 entities, preferably at least 5,000 entities, more preferably at least 10,000 entities, and even more preferably at least 50,000 entities.

As used herein, the term “target molecule” or “molecular target” refers to any kind of molecules, and in particular any kind of molecules which may be possibly present in a cell. The molecular target can be a biomolecule, i.e. a molecule that is naturally present in living organisms, or a chemical compound that is not naturally found in living organism such as pharmaceutical drugs, toxicants, heavy metals, pollutants, etc. . . . . Preferably, the molecular target is a biomolecule. Examples of biomolecules include, but are not limited to, nucleic acids, e.g. DNA or RNA molecules, proteins such as antibodies, enzymes or growth factors, lipids such as fatty acids, glycolipids, sterols or glycerolipids, vitamins, hormones, neurotransmitters, and carbohydrates, e.g., mono-, oligo- and polysaccharides. The terms “polypeptide”, “peptide” and “protein” are used interchangeably to refer to a polymer of amino acid residues, and are not limited to a minimum length. The protein may comprise any post-translational modification such as phosphorylation, acetylation, amidation, methylation, glycosylation or lipidation. As used herein, the term “nucleic acid” or “polynucleotide” refers to a polymeric form of nucleotides of any length, either ribonucleotides or deoxyribonucleotides.

One of the main advantage of the method of the invention, is the possibility to label, in a single run, different types of molecular targets such as proteins and nucleic acids. Thus, the term “plurality of molecular targets” may refer to different copies of the same molecule, e.g. different copies of the same mRNA or of the same protein, or may refer to different copies of different molecules, e.g. different copies of a mRNA and different copies of a protein.

In an embodiment, molecular targets are different copies of the same molecule. Preferably the molecule is biomolecule, more preferably a nucleic acid or a protein, even more preferably a RNA molecule or a protein. In another embodiment, molecular targets are different copies of different molecules. Preferably said molecules are biomolecules, more preferably are nucleic acids and/or proteins, even more preferably RNA molecules and/or proteins. In a particular embodiment, molecular targets are different copies of at least two different nucleic acids, preferably RNA. In another particular embodiment, molecular targets are different copies of at least two different proteins. In a further embodiment, molecular targets are different copies of one or several nucleic acid, preferably RNA, and different copies of one or several proteins.

In preferred embodiments, the method of the invention is implemented using one or several microfluidic systems, i.e. at least one step of the method is implemented using a microfluidic system. In some embodiments, the method is implemented using several microfluidic systems, for example a microfluidic system to generate the first set of emulsion droplets, a microfluidic system to generate the second set of emulsion droplets and a microfluidic system to fuse the two sets and optionally to conduct some subsequent steps. In a particular embodiment, one or several microfluidic systems are used to generate the first set of emulsion droplets, one or several microfluidic systems are used to generate the second set of emulsion droplets and a microfluidic system to fuse the two sets. In some other embodiments, the method is implemented using a microfluidic system wherein the first set of emulsion droplets and/or the second set of emulsion droplets are generated and wherein droplets of the two sets are fused.

As used herein, the terms “emulsion droplet”, “droplet” and “microfluidic droplet” are used interchangeably and may refer to a water-in-oil emulsion droplet (also named w/o droplet), i.e. an isolated portion of an aqueous phase that is completely surrounded by an oil phase, an oil-in-water emulsion droplet (also named o/w droplet), i.e. an isolated portion of an oil phase that is completely surrounded by an aqueous phase, a water-in-oil-in-water emulsion droplet (also named w/o/w droplet) consisting of an aqueous droplet inside an oil droplet, i.e. an aqueous core and an oil shell, surrounded by an aqueous carrier fluid, or an oil-in-water-in-oil emulsion droplet (also named o/w/o droplet) consisting of an oil droplet inside an aqueous droplet, i.e. an oil core and an aqueous shell, surrounded by an oil carrier fluid. Preferably, this term refers to a w/o emulsion droplet.

A droplet may be spherical or of other shapes depending on the external environment. Typically, the droplet has a volume of less than 100 nL, preferably of less than 10 nL, and more preferably of less than 1 nL. For instance, a droplet may have a volume ranging from 2 pL to 1 nL, preferably from 2 to 500 pL, more preferably from 2 to 100 pL. Preferably, the droplets have a homogenous distribution of diameters, i.e., the droplets may have a distribution of diameters such that no more than about 10%, about 5%, about 3%, about 1%, about 0.03%, or about 0.01% of the droplets have an average diameter greater than about 10%, about 5%, about 3%, about 1%, about 0.03%, or about 0.01% of the average diameter of the droplets. Preferably, the emulsion is a monodispersed emulsion, i.e. an emulsion comprising droplets of the same volume. Techniques for producing such a homogenous distribution of diameters are well-known by the skilled person (see for example WO 2004/091763).

The aqueous phase is typically water or an aqueous buffer solution, such as but not limited to Tris-HCl buffer, Tris-acetate buffer, phosphate buffer saline (PBS) or acetate buffer. Preferably, the aqueous phase is an aqueous buffer solution. Optionally, the aqueous phase may comprise bovine serum albumin or additive such as Pluronic. In preferred embodiments, the aqueous phase is chosen in order to be compatible with enzymatic reactions performed during the process of the invention, such as enzymatic digestion, amplification, ligation, etc. . . . . An example of such aqueous phase includes, but is not limited to, CutSmart restriction enzyme buffer (New England Biolabs).

The oil phase used to generate the emulsion droplets may be selected from the group consisting of fluorinated oil such as FC40 oil (3M®), FC43 (3M®), FC77 oil (3M®), FC72 (3M®), FC84 (3M®), FC70 (3M®), Novec-7500 (3M®), Novec-7100 (3M®), perfluorohexane, perfluorooctane, perfluorodecane, Galden-HT135 oil (Solvay Solexis), Galden-HT170 oil (Solvay Solexis), Galden-HT110 oil (Solvay Solexis), Galden-HT90 oil (Solvay Solexis), Galden-HT70 oil (Solvay Solexis), Galden PFPE liquids, Galden® SV Fluids or H-Galden® ZV Fluids; and hydrocarbon oils such as Mineral oils, Light mineral oil, Adepsine oil, Albolene, Cable oil, Baby Oil, Drakeol, Electrical Insulating Oil, Heat-treating oil, Hydraulic oil, Lignite oil, Liquid paraffin, Mineral Seal Oil, Paraffin oil, Petroleum, Technical oil, White oil, Silicone oils or Vegetable oils. Preferably, the oil phase is fluorinated oil such as Novec-7500, FC40 oil, Galden-HT135 oil or FC77 oil, more preferably is Novec-7500. The skilled person may easily select suitable phase oil to implement the methods of the invention.

The emulsion droplets comprise one or several surfactants. Said surfactant(s) can aid in controlling or optimizing droplet size, flow and uniformity and stabilizing aqueous emulsions. Suitable surfactants for preparing the emulsion droplets used in the present invention are typically non-ionic and contain at least one hydrophilic head and one or several lipophilic tails, preferably one (diblock surfactant) or two (triblock surfactant) lipophilic tails. Said hydrophilic head(s) and the tail(s) may be directly linked or linked via a spacer moiety. Examples of suitable surfactants include, but are not limited to, sorbitan-based carboxylic acid esters such as sorbitan monolaurate (Span 20), sorbitan monopalmitate (Span 40), sorbitan monostearate (Span 60) and sorbitan monooleate (Span 80); block copolymers of polyethylene glycol and polypropylene glycol such as the triblock copolymer EA-surfactant (RainDance Technologies), DMP (dimorpholino phosphate)-surfactant (Baret, Kleinschmidt, et al., 2009) and Jeffamine-surfactant; polymeric silicon-based surfactants such as Abil EM 90; triton X-100; and fluorinated surfactants such as PFPE-PEG and perfluorinated polyethers (e.g., Krytox-PEG, DuPont Krytox 157 FSL, FSM, and/or ESH). In the context of the invention, preferred surfactants are fluorinated surfactants, i.e. fluorosurfactants.

In some particular embodiments, the emulsion droplets comprise one or several functionalized surfactants at their interface. As used herein, a “functionalized surfactant” refers to a surfactant which bears at least one functional moiety either on one of its hydrophilic head(s) or lipophilic tail(s), preferably on a hydrophilic head. As used herein, a “functional moiety” is virtually any chemical or biological entity which provides the surfactant with a function of interest. For instance, the functional moiety can enable to create a covalent or non-covalent interaction between the surfactant and a molecular target of interest. Thanks to the use of such functionalized surfactants, molecular targets may be exposed on the surface of emulsion droplets. The interface of these droplets may comprise only functionalized surfactant(s) or a mix of functionalized and non-functionalized surfactants. The ratio between functionalized and non-functionalized surfactants may vary and can be easily adapted by the skilled person. For example, functionalized surfactant may represent from 1 to 100% (w/w) of total surfactants, preferably from 2 to 80% (w/w), and more preferably from 5 to 50% (w/w).

The total amount of surfactant in the carrier oil is preferably chosen in order to ensure stability of the emulsion and prevent spontaneous coalescence of droplets. Typically, the carrier oil comprises from 0.5 to 10% (w/w), preferably from 1 to 8% (w/w), and more preferably from 2 to 5% (w/w) of surfactant.

The emulsion can be prepared by any method known by the skilled artisan. Preferably, the emulsion can be prepared on a microfluidic system.

Providing the First Set of Emulsion Droplets Comprising Molecular Targets

The first set of emulsion droplets comprises droplets containing molecular targets, wherein each of these droplets comprises a plurality of molecular targets originating from no more than one entity. In preferred embodiments, these droplets do not contain any barcode which will be or can be subsequently used to label a molecular target. In particular, they do not contain any UMI barcode as defined below.

The first set of emulsion droplets may be a w/o or o/w/o emulsion depending on the nature of the entities. In some embodiments, the entities are cells or particles and emulsion droplets of the first set are w/o emulsion droplets. In some other embodiments, the entities are o/w emulsion droplets exposing molecular targets on their outer surfaces and emulsion droplets of the first set are o/w/o emulsion droplets.

Preferably, the first set of emulsion droplets comprises at least 10,000 droplets, preferably at least 100,000 droplets, preferably at least 500,000 droplets, and even more preferably at least 1,000,000 droplets.

In some embodiments, the first set of emulsion droplets is obtained by encapsulating entities within emulsion droplets, each droplet containing no more than one entity, and optionally lysing said entities within the droplets to release molecular targets.

In particular embodiments, entities are particles or o/w emulsion droplets exposing molecular targets on their outer surfaces, and the first set of emulsion droplets is obtained by encapsulating entities within emulsion droplets, each droplet containing no more than one entity.

In preferred embodiments, entities are cells and the first set of emulsion droplets is obtained by
encapsulating entities within emulsion droplets, each droplet containing no more than one entity, and
lysing said entities within the droplets to release molecular targets.

To ensure single-entity resolution, entities have to be confined and isolated from the beginning to the end of the barcoding process. Encapsulation of entities into microfluidic droplets is a convenient way to isolate said entities and is particularly suitable to high throughput regimes.

Those of ordinary skill in the art is aware of techniques for encapsulating cells or particles within microfluidic droplets (see, for example, the international patent application WO 2004/091763 incorporated herein by reference). If cells are adherent or from tissue, they may be first dissociated and optionally filtered or centrifuged to remove clumps of two or more cells before encapsulation. Cells may typically be suspended in an aqueous buffer such as PBS buffer.

Methods for producing monodisperse w/o/w double emulsions, i.e. for encapsulating o/w droplets are also well known by the skilled person and microfluidic systems generating such emulsions are commercially available.

During encapsulation, the entity number density (entities per unit volume) has to be adjusted to minimize incidences of two or more entities becoming captured in the same droplet. In particular, entities may be encapsulated at a density of less than 1 entity per droplet, preferably at a density of less than 0.2 entity per droplet, in order to prevent co-encapsulation of two or more entities. In preferred embodiments, the entity number density and the average occupancy is adjusted in order to ensure that most, preferably at least 98%, or all of the droplets have only zero or one entity present in them.

Using any well-known microfluidic method to encapsulate entities into microfluidic droplets, entity-bearing droplets may be produced at high frequency, e.g. ranging from 0.5 kHz to 15 kHz, preferably from 1 kHz to 10 kHz, more preferably from 1 kHz to 5 kHz.

In embodiments wherein entities are cells, after encapsulation, cells may be lysed within the droplets in order to release molecular targets. Cell lysis may be performed using any method known by the skilled person such as using physical, chemical or biological means. In particular, cells may be lysed using radiation (e.g. UV, X or γ-rays), laser (see e.g. Rau et al., 2004) or an electric field (de Lange et al., 2016). The lysis may also be induced by osmotic shock or by addition of a detergent or enzyme (see, e.g. Kintses et al., 2012; Novak et al., 2011; Brown & Audet, 2008). The lysis may also be induced by heat shock.

In some embodiments, the lysis is induced by a lysis agent. Preferably, the lysis agent comprises one or several components altering the osmotic balance, one or several detergents and/or one or several enzymes. More preferably, the lysis agent is Triton X-100, BugBuster® reagent (Merck Millipore), Nonidet P40™ (MP BioMedical), M-PER™ (Thermo Scientific) or B-PER™ (Thermo Scientific).

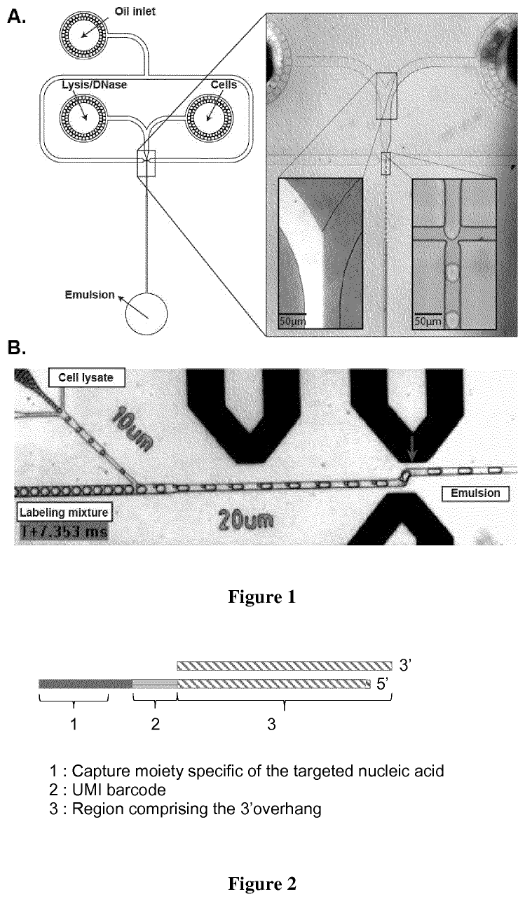

In an embodiment, the lysis agent is directly added to the aqueous phase of the droplets before encapsulation. In such embodiment, an aqueous stream containing the cells may be combined with a stream of aqueous solution containing the lysis agent just before generation of droplets (see, e.g. FIG. 1A). The emulsion may be then generated, collected and incubated to allow cell lysis.

In another embodiment, the lysis agent is introduced inside the droplet after droplet generation by any known technique such as pico-injection or droplet fusion. The emulsion may be then collected and incubated to allow cell lysis.

Typically, the emulsion is incubated from 5 minutes to 1 hour and at a temperature ranging from 4° C. to 25° C. to allow cell lysis.

Alternatively, the lysis may be induced by a heat treatment. Typically, in this case, the emulsion may be incubated from 5 minutes to 1 hour and at a temperature up to 95° C. to allow cell lysis.

The skilled person can easily adapt the incubation temperature during the lysis to the used method.

In some embodiments wherein the w/o interface of the first set droplets comprises functionalized surfactant(s), some or all molecular targets released by cell lysis, may be bound by said surfactant(s) and concentrated onto the inner w/o interface of droplets. Possibly, these w/o droplets can be convert into o/w droplets using droplet inversion as presented in international patent application WO 2017/174610.

In some embodiments wherein entities are particles or o/w emulsion droplets exposing molecular targets on their outer surfaces, molecular targets can be released from said particles or from the surface of said o/w emulsion droplets by the action of a cleaving agent (e.g. restriction enzyme).

Optionally, depending on the nature of molecular targets, one or several additional reagents may be added to the aqueous phase before collection and incubation of the first set emulsion. Examples of such additional reagents may include, but are not limited to DNases, RNases, proteases, protease inhibitors and/or nuclease inhibitors.

In some embodiments, molecular targets are RNA and one or several additional reagents, preferably comprising one or several DNases and/or one or several proteases, are added to the aqueous phase. In some other embodiments, molecular targets are proteins and/or RNA and additional reagents, preferably comprising one or several DNases, are added to the aqueous phase.

Additional reagent(s) and lysis agent may be added simultaneously to the aqueous phase, i.e. directly added to the aqueous phase of the droplets just before encapsulation or after droplet generation by any known technique such as pico-injection or droplet fusion. Alternatively, additional reagent(s) and lysis agent may be added sequentially. In particular, the lysis agent may be added to the aqueous phase before encapsulation by co-flowing a flow of an aqueous solution containing the entities and a flow of a solution containing the lysis agent, and additional reagent(s) may be added after encapsulation, and vice-versa, or the lysis agent and additional reagent(s) may be added sequentially after encapsulation, e.g. separate pico-injection or droplet fusion.

In preferred embodiments, additional reagent(s) and lysis agent are added simultaneously to the aqueous phase, i.e. directly added to the aqueous phase of the droplets just before encapsulation (see, e.g. FIG. 1A).

Providing the Second Set of Emulsion Droplets Comprising Cell Identification Sequences

The second set of emulsion droplets comprises droplets containing probes.

After fusion of droplets of the first and second sets, these probes can specifically detect and label molecular targets contained in droplets of the first set (see, e.g. FIG. 1B).

Each probe comprises a capture moiety capable of specific binding or ligation to a molecular target contained in droplets of the first set or to an adaptor linked to said molecular target, and a DNA moiety comprising an identification sequence.

The specific detection of molecular targets relies on the capture moiety whereas the barcoding of molecular targets relies on the DNA identification sequence which comprises a molecular identification (UMI) barcode, an entity identification (UEI) barcode and a calibrator (UEI-calibrator) barcode.

In particular, the method of the invention further comprises

encapsulating a plurality of entity identification (UEI) sequences, a plurality of calibrator (UEI-calibrator) sequences and a plurality of molecular identification (UMI) molecules with an amplification reaction mixture within emulsion droplets.

wherein each droplet comprising one or several UEI sequences, one or several UEI-calibrator sequences and a plurality of UMI molecules, the combination of UEI sequences and UEI-calibrator sequences being different for each droplet and each droplet comprising a plurality of UMI molecules,

wherein each UEI sequence comprises a UEI barcode and one or two overhang producing restriction sites,

each UEI-calibrator sequence comprises a UEI-calibrator barcode and one or two overhang producing restriction sites,

each UMI molecule comprises a capture moiety capable of specific binding or ligation to a molecular target or to an adaptor linked to said molecular target, and a DNA moiety comprising (i) a region proximal to the capture moiety and comprising a UMI barcode and (ii) a region distal from the capture moiety and comprising an overhang or an overhang producing restriction site, and

each UMI molecule comprises a UMI barcode which is different from the other UMI molecules contained in the same droplet;

amplifying UEI sequences and UEI-calibrator sequences within droplets; and

assembling UEI-calibrator barcodes, UEI barcodes and UMI molecules through restriction enzyme digestion and ligation of compatible overhangs,

thereby obtaining the second set of emulsion droplets.

Assembling UEI, UEI-calibrator and UMI barcodes leads to the formation of different chimeras between UEI and UEI-calibrator barcodes. These pairs of sequences constitute a signature unique to each droplet and allow reassigning each analyzed molecule to its original entity, despite the presence of several UEI sequences.

Unique Molecular Identification (UMI) Molecules

Each UMI molecule comprises
a capture moiety capable of specific binding or ligation to a molecular target or to an adaptor linked to said molecular target, and
a DNA moiety comprising (i) a region proximal to the capture moiety and comprising a UMI barcode and (ii) a region distal from the capture moiety and comprising an overhang or an overhang producing restriction site

As used herein, the term “UMI barcode” refers to a randomized nucleotide sequence assigning a unique barcode to each molecular target contained in a droplet of the first set and thus allows further performing the digital detection/counting of molecular targets initially present into or onto the entity, their absolute quantification and correcting for amplification biases. Indeed, in a droplet, each UMI molecule carries a unique identification number, i.e. the UMI barcode, and therefore counting the number of different UMI barcodes gives the absolute number of molecular targets initially present into or onto the entity.

Preferably, the UMI barcode is a randomized nucleotide sequence having a length of at least 5 nucleotides, preferably a length from 5 to 15 nucleotides, more preferably a length from 5 to 10 nucleotides. The randomized sequence can be a stretch of contiguous randomized nucleotides or a stretch of semi-randomized nucleotides (i.e. contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UMI barcode is a stretch of semi-randomized nucleotides, in particular a stretch where several randomized dinucleotides are spaced by constant dinucleotides.

Optionally, the UMI barcode may further comprise a type identifier sequence which is a short pre-defined sequence, preferably having a length from 4 to 8 nucleotides, coding for the nature (e.g. nucleic acid or protein) and/or the identity (e.g. GFP mRNA) of the molecular target.

In the region distal from the capture moiety, the UMI molecule comprises an overhang or an overhang producing restriction site. Indeed, an overhang is required to assemble the identification sequence, i.e. to assemble UMI molecules with UEI and UEI-calibrator sequences. This overhang may be a 3′overhang or a 5′overhang, preferably is a 3′overhang.

The UMI molecule may comprise an overhang (3′ or 5′ overhang) compatible with a cohesive end generated by a restriction enzyme or may comprise an overhang-producing restriction site.

In some embodiments, the UMI molecule comprises an overhang, preferably a 3′ overhang, compatible with a cohesive end generated by a restriction enzyme. The choice of this configuration ensures that no complementary strand will be synthesized by the filling activity of a polymerase so that the extremity will stay competent for the addition of UEI and UEI-calibrator sequences. In addition, this overhang is compatible with the cleavage product of the restriction enzyme which is used to digest UEI and/or UEI-calibrator sequences detailed below. Using digestion and ligation, this overhang allows the addition of UEI and UEI-calibrator sequences to each UMI molecule.

In some other embodiments, the UMI molecule comprises an overhang producing restriction site, i.e. a restriction site generating a 3′ or 5′overhang, preferably 3′overhang, which is compatible with the cleavage product of the restriction enzyme which is used to digest UEI and/or UEI-calibrator sequences. In a particular embodiment, the overhang producing restriction site on the UMI molecule is recognized by the same enzyme than the overhang producing restriction site on UEI and/or UEI-calibrator sequences (see below).

In preferred embodiments, each UMI molecule comprises

a capture moiety capable of specific binding to a molecular target or specific ligation to a molecular target or an adaptor linked to said molecular target and

a DNA moiety comprising (i) a region proximal to the capture moiety and a UMI barcode, and (ii) a region distal from the capture moiety and comprising an overhang, preferably an overhang compatible with a cohesive end generated by a restriction enzyme, or an overhang producing restriction site. Preferably the region distal from the capture moiety comprises a 3′overhang or a 3′overhang producing restriction site.

In embodiments wherein the region distal from the capture moiety comprises a 3′overhang, the 5′ end is preferably a phosphorylated 5′ end.

Preferably, the restriction site or overhang is separated from the UMI barcode by at least 10 nucleotides, preferably at least 20 nucleotides, and more preferably 20 to 40 nucleotides. Preferably, this separating region has a melting temperature of at least 50° C., more preferably at least 55° C., is GC rich in order to form stable duplexes, does not exhibit any sequence identity with a nucleic acid found in the organism from which the cell is originated and does not contain one of the restriction sites later used in the labelling process. In preferred embodiments, this region is identical for all probes.

In a particular embodiment, the DNA moiety comprises, from the capture moiety to the restriction site or overhang, (i) UMI barcode comprising a type identifier sequence of 4 to 8 nucleotides, preferably of 4 nucleotides, and a barcode of 5 to 15 nucleotides, preferably of 8 nucleotides, and (ii) a region of 20 to 40 nucleotides comprising the restriction site or overhang.

The nature of the capture moiety and the structure of the DNA moiety may differ according to type and/or the chemical structure of molecular targets. Since molecular targets of different types and/or chemical structures may be labelled simultaneously, probes contained in the same droplet may have different structures.

In some embodiments, at least some of molecular targets are nucleic acids and at least some UMI molecules specific of said nucleic acids are DNA UMI molecules comprising

a capture moiety which is a single stranded DNA region which drives the specific recognition of a nucleic acid molecular target, or the specific recognition of a nucleic acid adaptor linked to said molecular target, through conventional Watson-Crick base-pairing interactions, and

a DNA moiety comprising (i) a 3′ single stranded region proximal to the capture moiety and comprising the UMI barcode and (ii) a 5′ double-stranded region distal from the capture moiety and comprising an overhang, preferably an overhang compatible with a cohesive end generated by a restriction enzyme, or an overhang producing restriction site.

Preferably, the DNA moiety comprises (i) a 3′ single stranded region proximal to the capture moiety and comprising the UMI barcode and (ii) a 5′ double-stranded region distal from the capture moiety and comprising a 3′overhang, preferably compatible with a cohesive end generated by a restriction enzyme.

These DNA UMI molecules may be produced by any method known by the skilled person such as chemical synthesis.

An exemplary embodiment of a DNA UMI molecule is presented in FIG. 2.

In these embodiments, the length of the capture moiety has to be sufficient to allow the specific recognition of the target molecule through hybridization. Preferably, the capture moiety is a single stranded DNA region of at least 8 nucleotide long, preferably of 8 to 25 nucleotide long, more preferably of 10 to 15 nucleotide long.

The melting temperature (Tm) of the perfect hybrid formed upon association of the capture moiety with the molecular target is preferably adjusted (e.g. by modulating the length of the sequence specific to the target nucleic acid) in order to be ranged between 30° C. and 70° C., preferably between 30° C. and 60° C., more preferably between 40° C. and 60° C., and even more preferably between 40° C. and 50° C.

Preferably, the difference between melting temperature (Tm) of all capture moieties specific to nucleic acid molecular targets, is lower than 3° C., more preferably lower than 2° C. and even more preferably lower than 1° C.

The capture moiety may be specific to a particular DNA or RNA or may be complementary to a sequence region common to all RNAs, e.g. the capture moiety may be a poly-T tract which is complementary to the poly-A tails of eukaryotic mRNAs or complementary to a nucleic acid adaptor, preferably a DNA adaptor, added to all RNA, for instance through the action of an RNA ligase.

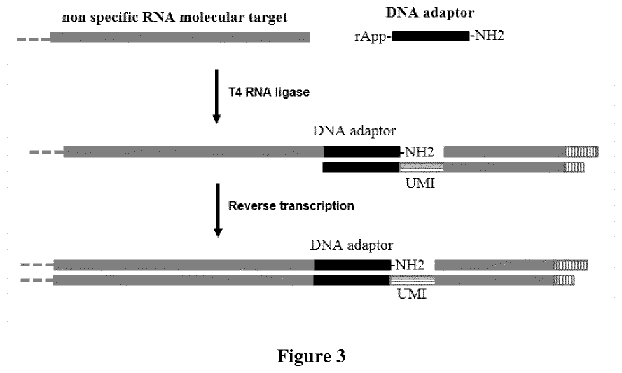

Thus, in some embodiments, the capture moiety drives the specific recognition of a nucleic acid adaptor linked to the molecular targets of interest, e.g. all RNAs. Such adaptor may be, for example, pre-adenylated oligonucleotides (5′-App oligos) which act as substrates for T4 ligases and thus can be ligated to any RNA molecule. Typically, such adaptor may be a single stranded DNA region of at least 8 nucleotide long, preferably of 8 to 25 nucleotide, more preferably of 10 to 15 nucleotide long. Such adaptors may be provided in droplets of the first set or in droplets of the second set. An exemplary illustration of such embodiment is presented in FIG. 3.

In some other embodiments, at least some of molecular targets are nucleic acids and at least some UMI molecules specific of said nucleic acids are DNA UMI molecules comprising

a capture moiety which is a single stranded DNA region which is able to ligate to a nucleic acid molecular target, and

a DNA moiety comprising (i) a 5′ single stranded region proximal to the capture moiety and comprising the UMI barcode and (ii) a 3′ double-stranded region distal from the capture moiety and comprising an overhang, preferably an overhang compatible with a cohesive end generated by a restriction enzyme, or an overhang producing restriction site.

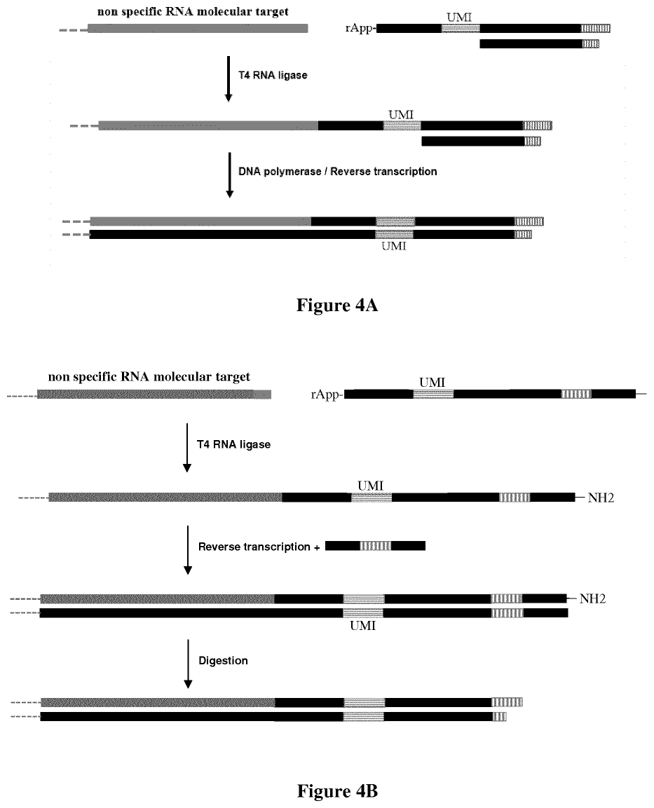

Preferably, the capture moiety is a 5′ single stranded DNA region comprising 5′,5′-adenyl pyrophosphoryl moiety (App) onto its 5′-end. Such moiety may act as substrate for T4 ligases and thus can be ligated to the 3′ end any RNA molecule. In this embodiment, the 5′ App extremity of the capture moiety directly interacts with the RNA molecular target and ligates to said target in the presence of T4 ligase. Exemplary illustrations of such embodiment are presented in FIGS. 4A and 4B.

These DNA UMI molecules may be produced by any method known by the skilled person such as chemical synthesis.

Alternatively, or additionally, at least some UMI molecules may be chimeric molecules made of synthetic DNA oligonucleotides, i.e. the DNA moiety, covalently associated to a second molecule, i.e. the capture moiety, targeting specifically and with high affinity the target molecule and making possible to specifically label any molecule with a signal amplifiable and readable. These capture moieties may be specific of any type of molecular targets, preferably are specific of protein molecular targets.

The capture moiety may be

(i) a binding moiety that specifically binds to a molecular target and is directly bound to the DNA moiety,

(ii) a chimeric protein comprising a first domain that specifically binds to a molecular target and a second domain that binds to the DNA moiety, or

(iii) a binding moiety that binds specifically to a molecular target and a protein bridge, said protein bridge comprising a first domain that binds to the binding moiety and a second domain that binds to the DNA moiety.

The term “specifically binding” or “specifically binds” is used herein to indicate that this moiety has the capacity to recognize and interact specifically with the molecular target of interest, while having relatively little detectable reactivity with other structures present in the aqueous phase such as other molecular targets that can be recognized by other probes. There is commonly a low degree of affinity between any two molecules due to non-covalent forces such as electrostatic forces, hydrogen bonds, Van der Waals forces and hydrophobic forces, which is not restricted to a particular site on the molecules, and is largely independent of the identity of the molecules. This low degree of affinity can result in non-specific binding. By contrast when two molecules bind specifically, the degree of affinity is much greater than such non-specific binding interactions. In specific binding a particular site on each molecule interacts, the particular sites being structurally complementary, with the result that the capacity to form non-covalent bonds is increased. Specificity can be relatively determined by binding or competitive assays, using e.g., Biacore instruments. The affinity of a molecule X for its partner Y can generally be represented by the dissociation constant (Kd). In preferred embodiments, the Kd representing the affinity between the capture moiety and the molecular target of interest is from 1·10⁻⁷M or lower, preferably from 1·10⁻⁸M or lower, and even more preferably from 1·10⁻⁹M or lower.

In some embodiments, at least some UMI molecules comprise a capture moiety which is a binding moiety that specifically binds to a molecular target and is directly bound to the DNA moiety. Preferably the binding moiety is covalently bound to the DNA moiety.

Examples of binding moieties include, but are not limited to, antibodies, ligands of ligand/anti-ligand couples, peptide and nucleic acid aptamers, protein tags, or chemical probes (e.g. suicide substrate) reacting specifically with a molecular target or a class of molecular targets.

Examples of ligand/anti-ligand couples include, but are not limited to, antibody/antigen or ligand/receptor. In particular, in some embodiments, the molecular target is an antibody and the binding moiety is an antigen recognized by said antibody, or vice-versa. In some other embodiments, the molecular target is a receptor and the binding moiety is a ligand recognized by said receptor, or vice-versa.

A multitude of protein tags are well-known by the skilled person (see for example Young et al. Biotechnol. J. 2012, 7, 620-634) and may be used in the present invention. Examples of such protein tags include, but are not limited to, biotin (for binding to streptavidin or avidin derivatives), glutathione (for binding to proteins or other substances linked to glutathione-S-transferase), lectins (for binding to sugar moieties), c-myc tag, hemaglutinin antigen (HA) tag, thioredoxin tag, FLAG tag, polyArg tag, polyHis tag, Strep-tag, OmpA signal sequence tag, calmodulin-binding peptide, chitin-binding domain, cellulose-binding domain, S-tag, and Softag3, and the like.

A multitude of chemical probes are well-known by the skilled person (see for example Niphakis and Cravatt, Ann. Rev. of Biochem. 2014, 83, 341-77 and Willems et al. Bioconjugate Chem. 2014, 25, 1181-91) and may be used in the present invention. Examples of chemical probes include, but are not limited to, electrophile or photoreactive Activity-Based Probes (ABP), suicide substrate-based ABP and inhibitors-based ABP.

In a particular embodiment, at least some UMI molecules comprise a nucleic acid or peptide aptamer as capture moiety. Similar to antibodies, aptamers interact with their targets by recognizing a specific three-dimensional structure. Aptamers can specifically recognize a wide range of targets, such as proteins, nucleic acids, ions or small molecules such as drugs and toxins.

Peptides aptamers consist of a short variable peptide loop attached at both ends to a protein scaffold such as the bacterial protein thioredoxin-A. Typically, the variable loop length is composed of ten to twenty amino acids. Peptide aptamer specific of a target of interest may be selected using any method known by the skilled person such as the yeast two-hybrid system or Phage Display. Peptides aptamers may be produced by any method known by the skilled person such as chemical synthesis or production in a recombinant bacterium followed by purification.

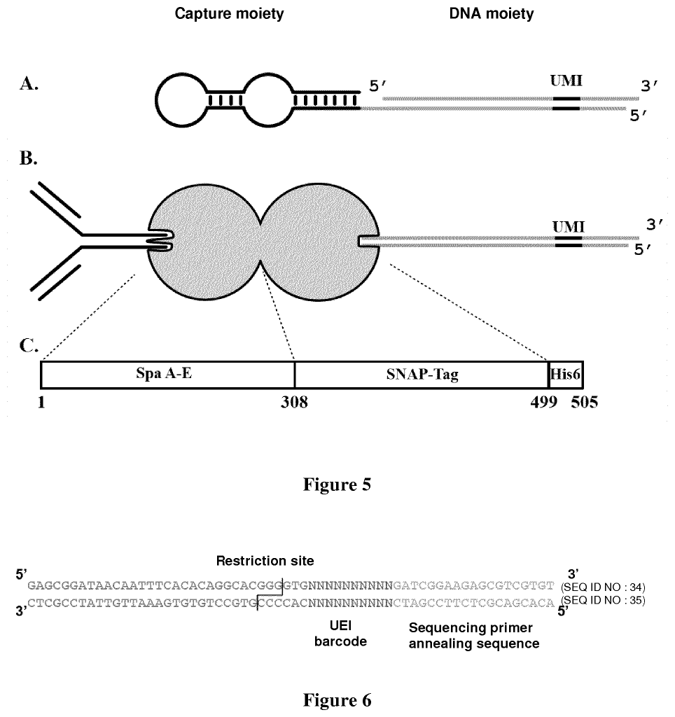

Preferably, at least some UMI molecules comprise a nucleic acid aptamer as capture moiety. Nucleic acid aptamers are a class of small nucleic acid ligands that are composed of RNA or single-stranded DNA oligonucleotides and have high specificity and affinity for their targets. Systematic Evolution of Ligands by EXponential enrichment (SELEX) technology to develop nucleic acid aptamers specific of a target of interest, is well known by the skilled person and may be used to obtain aptamers specific of a particular molecular target. Nucleic acid aptamers may be produced by any method known by the skilled person such as chemical synthesis or in vitro transcription for RNA aptamers. Preferably, nucleic acid aptamers used as capture moiety are selected from the group consisting of DNA aptamers, RNA aptamers, XNA aptamers (nucleic acid aptamer comprising xeno nucleotides) and spiegelmers (which are composed entirely of an unnatural L-ribonucleic acid backbone). An exemplary illustration of such a probe is presented in FIG. 5A.

In another particular embodiment, at least some UMI molecules comprise an antibody as capture moiety.

The term “antibody” herein is used in the broadest sense and specifically covers monoclonal antibodies, polyclonal antibodies, antibody fragments, and derivatives thereof, so long as they specifically bind to the molecular target of interest. In particular, the antibody may be a full length monoclonal or polyclonal antibody, preferably a full length monoclonal antibody. Preferably, this term refers to an antibody with heavy chains that contain an Fc region. By “Fc”, “Fc fragment” or “Fc region”, used herein is meant the polypeptide comprising the constant region of an antibody excluding the first constant region immunoglobulin domain. Thus, Fc refers to the last two constant region immunoglobulin domains of IgA, IgD, and IgG, and the last three constant region immunoglobulin domains of IgE and IgM, and the flexible hinge N-terminal to these domains. Preferably, the antibody is a full length monoclonal or polyclonal IgG antibody, preferably a full length monoclonal IgG antibody. A large number of specific and high affinity monoclonal antibodies are currently available on the market.

As used herein, the term “antibody fragment” refers to a protein comprising a portion of a full-length antibody, generally the antigen binding or variable domain thereof. Examples of antibody fragments include Fab, Fab′, F(ab)₂, F(ab′)₂, F(ab)₃, Fv (typically the VL and VH domains of a single arm of an antibody), single-chain Fv (ScFv), dsFv, Fd (typically the VH and CH1 domains) and dAb (typically a VH domain) fragments, nanobodies, minibodies, diabodies, triabodies, tetrabodies, kappa bodies, linear antibodies, and other antibody fragments that retain antigen-binding function (e.g. Holliger and Hudson, Nat Biotechnol. 2005 September; 23(9):1126-36). Antibody fragments can be made by various techniques, including but not limited to proteolytic digestion of intact antibody as well as recombinant host cells (e.g. E. coli or phage). These techniques are well-known by the skilled person and are extensively described in the literature. Preferably, the antibody fragment is selected from the group consisting of Fab′, F(ab)₂, F(ab′)₂, F(ab)₃, Fv, single-chain Fv (ScFv) fragments and nanobodies.

The term “antibody derivative”, as used herein, refers to an antibody provided herein, e.g. a full-length antibody or a fragment of an antibody, wherein one or more of the amino acids are chemically modified, e.g. by alkylation, PEGylation, acylation, ester or amide formation or the like. In particular, this term may refer to an antibody provided herein that is further modified to contain additional nonproteinaceous moieties that are known in the art and readily available.

In some embodiments, the capture moiety is selected from the group consisting of monoclonal and polyclonal antibodies, Fab′, F(ab)₂, F(ab′)₂, F(ab)₃, Fv, single-chain Fv (ScFv) fragments and nanobodies, and derivatives thereof. In some preferred embodiments, the capture moiety is selected from the group consisting of a monoclonal antibody, a ScFv fragment or a nanobody.

In some embodiments, at least some UMI molecules comprise a capture moiety which is a chimeric protein comprising a first domain that specifically binds to a single molecular target and a second domain that binds to a single DNA moiety. Preferably the second domain is covalently bound to the DNA moiety.

The first domain of the chimeric protein specifically binds to a single molecular target. Examples of first domains include, but are not limited to, antibodies, ligands of ligand/anti-ligand couples, peptide and nucleic acid aptamers, protein tags, and chemical probes, as described above. Preferably, the first domain of the chimeric protein is selected from the group consisting of antibodies and peptide aptamers, more preferably is a monoclonal antibody.

In a particular embodiment, the first domain of the chimeric protein is an antibody, preferably selected from the group consisting of monoclonal and polyclonal antibodies, Fab′, F(ab)₂, F(ab′)₂, F(ab)₃, Fv, single-chain Fv (ScFv) fragments and nanobodies, and derivatives thereof. More preferably, the first domain of the chimeric protein is selected from the group consisting of a monoclonal antibody, a ScFv fragment or a nanobody, and even more preferably from the group consisting of a ScFv fragment or a nanobody.

The second domain that covalently binds to the DNA moiety may be any domain allowing covalently grafting of a single nucleic acid. Examples of such domains include, but are not limited to, SNAP-Tag® (New England Biolabs), CLIP-Tag® (New England Biolabs), Halo-Tag® (Promega). Preferably, the second domain is a SNAP-Tag®. The SNAP-tag is a 20 kDa mutant of the DNA repair protein O⁶-alkylguanine-DNA alkyltransferase that reacts specifically and rapidly with benzylguanine (BG) derivatives leading to irreversible covalent association of the SNAP-tag with the DNA moiety attached to BG.

The chimeric protein used as capture moiety and comprising the first and second domains may be produced as fusion protein using any well-known recombinant engineering technology, before to be covalently associated to the DNA moiety.

In some embodiments, at least some UMI molecules comprise a capture moiety comprising (i) a binding moiety that specifically binds to a molecular target and (ii) a protein bridge, said protein bridge comprising a first domain that binds to the binding moiety and a second domain that binds to the DNA moiety. Preferably the second domain is covalently bound to the DNA moiety. The first domain may be covalently or non-covalently bound to the binding moiety, preferably non-covalently bound. In embodiments wherein the first domain non-covalently binds to the binding moiety, the non-covalent interaction is preferably turned into covalent interaction by cross-linking the first domain and the binding moiety.

The second domain of the protein bridge may be as described above for the chimeric protein, i.e. any domain allowing covalently grafting of a single nucleic acid. Preferably, the second domain of the protein bridge is a SNAP-Tag®.

The first domain of the protein bridge may be any domain allowing covalent or non-covalent interaction with the binding moiety, preferably non-covalent interaction. Examples of such domain includes, but are not limited to, immunoglobulin-binding bacterial proteins such as protein A, protein A/G, protein G and protein L.

In an embodiment, the first domain of the protein bridge is an immunoglobulin-binding bacterial protein and the binding moiety is an antibody, preferably an antibody containing a Fc region, more preferably a full length monoclonal or polyclonal IgG antibody, preferably a full length monoclonal IgG antibody. The immunoglobulin-binding bacterial protein is preferably selected from protein A, protein A/G, protein G and protein L, one or several IgG-binding domains thereof, and functional derivatives thereof.

Protein A is a cell surface protein found in Staphylococcus aureus. It has the property of binding the Fc region of a mammalian antibody, in particular of IgG class antibodies. The amino-terminal region of this protein contains five highly homologous IgG-binding domains (termed E, D, A, B and C), and the carboxy terminal region anchors the protein to the cell wall and membrane. All five IgG-binding domains of protein A bind to IgG via the Fc region and in principle, each of these domains is sufficient for binding to the Fc-portion of an IgG.

Thus, in a particular embodiment, the first domain of the protein bridge is selected from the group consisting of domains A, B, C D and E of protein A, combinations thereof and functional derivatives thereof retaining IgG binding functionality of wild-type protein A. Preferably, the first domain of the protein bridge comprises domains A to E of protein A.

The protein bridge may be produced as fusion protein using any well-known recombinant engineering technology, before to be covalently associated to the DNA moiety.

Preferably, the first domain is located at the N-terminal part of the protein bridge and the second domain is located at the C-terminal part of the protein bridge.

Optionally, the protein bridge may further comprise at the C-terminal extremity an affinity tag (e.g. a polyhistidine-tag) to facilitate its purification.

In a particular embodiment, the protein bridge comprises an immunoglobulin-binding bacterial protein, preferably domains A to E of protein A, as first domain, a SNAP-Tag® as second domain and a monoclonal or polyclonal IgG antibody as binding moiety, preferably a monoclonal IgG antibody. An exemplary illustration of such a probe is presented in FIGS. 5B and C.

The binding moiety may be covalently or non-covalently bound to the protein bridge. In some embodiments, the protein bridge can be cross-linked with the binding moiety to ensure long-term physical link.

In chimeric UMI molecules described above, the DNA moiety comprises a double stranded DNA region comprising an overhang, preferably compatible with a cohesive end generated by a restriction enzyme, or an overhang producing restriction site as described above for DNA probes. Preferably, the overhang comprised in the DNA moiety or generated by the restriction site is a 3′overhang. As described above, the DNA moiety comprises a UMI barcode. The region of the DNA moiety comprising the UMI barcode may be a single stranded or double stranded region.

Preferably, in chimeric UMI molecules, the DNA moiety further comprises a sequencing primer annealing sequence which is proximal to the capture moiety. After labelling of molecular targets, this sequence allows direct amplification using sequencing primers.

Unique Entity Identification (UEI) Sequences and UEI-Calibrator Sequences

The UEI sequence is a linear or circular double stranded DNA sequence of 40 to 100 nucleotide long, preferably of 50 to 70 nucleotide long, comprising a UEI barcode and one or two overhang producing restriction sites, preferably one or two 3′overhang producing restriction site.

The “UEI barcode” is a randomized nucleotide sequence designed to identify molecular targets originating from the same entity. Preferably, the UEI barcode is a randomized nucleotide sequence having a length of at least 8 nucleotides, preferably a length from 8 to 20 nucleotides, more preferably a length from 8 to 15 nucleotides. The randomized sequence can be a stretch of contiguous randomized nucleotides or a stretch of semi-randomized nucleotides (i.e.

contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UEI barcode is a stretch of semi-randomized nucleotides, in particular a stretch where several randomized dinucleotides are spaced by constant dinucleotides.

The restriction sites comprised in the UEI sequence may generate, upon digestion with the corresponding restriction enzyme, an overhang compatible with the overhangs of UMI molecules and/or an overhang compatible with the overhangs of UEI-calibrator sequences as described below, and thus allows assembling of UEI sequences to UMI molecules and to UEI-calibrator sequences or assembling of UEI sequences to UEI-calibrator sequences (which are attached on the other side to the DNA moiety of UMI molecules). Preferably, this restriction site is a non-palindromic cleavage site.

In embodiments wherein the UEI sequence is or is intended to be at the extremity of the identification sequence, the UEI sequence may further comprise a sequencing primer annealing sequence adjacent to the UEI barcode and at the opposite end of the restriction site.

In preferred embodiments, the UEI sequence comprises a constant region allowing for amplification of the UEI sequence, adjacent to the restriction site and at the opposite end of the UEI barcode and the sequencing primer annealing sequence when present. Preferably, this constant region has a length of 15 to 35 nucleotides, preferably of 20 to 30 nucleotides. Preferably, this constant region has a melting temperature comprised between 50° C. and 70° C.

An exemplary illustration of such UEI sequence is presented in FIG. 6.

Preferably, all UEI sequences have to same sequence except for the UEI barcode, i.e. they exhibit the same sequencing primer annealing sequence, the same restriction site and the same constant region.

In order to increase droplet occupancy, encapsulation of several UEI sequences in one droplet is tolerated. The inventors found that single-entity resolution can be preserved by using UEI-calibrator sequences in addition to UEI sequences. Indeed, the possibility of co-encapsulating several UEI sequences in the same droplets leads to a dramatic increase of droplet occupancy. As illustration, adjusting the dilution of the UEI sequence solution in order to co-encapsulate five UEI sequences per droplets, leads to a droplet occupancy close to 100%.

Preferably, dilutions of UEI sequence solution and UEI-calibrator sequence solution are adjusted in order to co-encapsulate 2 to 10 UEI sequences per droplet, preferably 4 to 6 UEI sequences per droplet, more preferably 4 UEI sequences per droplet, and 2 to 10 UEI-calibrator sequences per droplet, preferably 4 to 6 UEI-calibrator sequences per droplet, more preferably 4 UEI-calibrator sequences per droplet.

UEI-calibrator sequences are linear or circular DNA sequences, preferably double stranded DNA sequences, comprising a UEI-calibrator barcode which is different for each UEI-calibrator sequence, and one or two overhang producing restriction sites, preferably generating overhangs compatible with overhangs of digested UEI sequences and/or UMI molecules.

Preferably, these overhang producing restriction sites are non-palindromic cleavage sites. Preferably, these restriction sites generate 3′ overhangs. The “UEI-calibrator barcode” is a randomized nucleotide sequence having a length of at least 15 nucleotides, preferably a length from 15 to 40 nucleotides, more preferably a length from 15 to 20 nucleotides. The randomized sequence can be a stretch of contiguous randomized nucleotides or a stretch of semi-randomized nucleotides (i.e. contiguous randomized nucleotides spaced by constant nucleotides). Typical examples of a stretch of semi-randomized nucleotides are stretches where several randomized dinucleotides are spaced by constant dinucleotides, or stretches where several randomized trinucleotides are spaced by constant trinucleotides. Preferably, the UEI-calibrator barcode is a stretch of semi-randomized nucleotides, in particular a stretch where several randomized dinucleotides are spaced by constant dinucleotides.

In an embodiment, UEI-calibrator sequences comprise one overhang producing restriction site which generates, upon digestion with the corresponding restriction enzyme, an overhang compatible with overhangs of digested UEI sequences comprising UEI barcodes.

Optionally, in this embodiment, UEI-calibrator sequences may further comprise a sequencing primer annealing sequence adjacent to the UEI-calibrator barcode and at the opposite end of the restriction site generating overhangs compatible with digested UEI sequences.

Optionally, in this embodiment, UEI-calibrators may further comprise a binding tag allowing specific capture of the molecule at the extremity proximal to the sequencing primer annealing sequence. Preferably, the binding tag is selected from biotin or digoxigenin, more preferably is biotin.

Preferably, in this embodiment, UEI-calibrators comprise a constant region adjacent to the restriction site and at the opposite end of the UEI-calibrator barcode and the sequencing primer annealing sequence when present. This constant region allows amplifying UEI-calibrator sequences, preferably using PCR amplification. In particular, this region may comprise a primer binding site. Preferably, this constant region has a length of 10 to 35 nucleotides, preferably of 15 to 25 nucleotides. Preferably this constant region is orthogonal to that of UEI or UMI sequences in order to prevent unwilling hybridization.

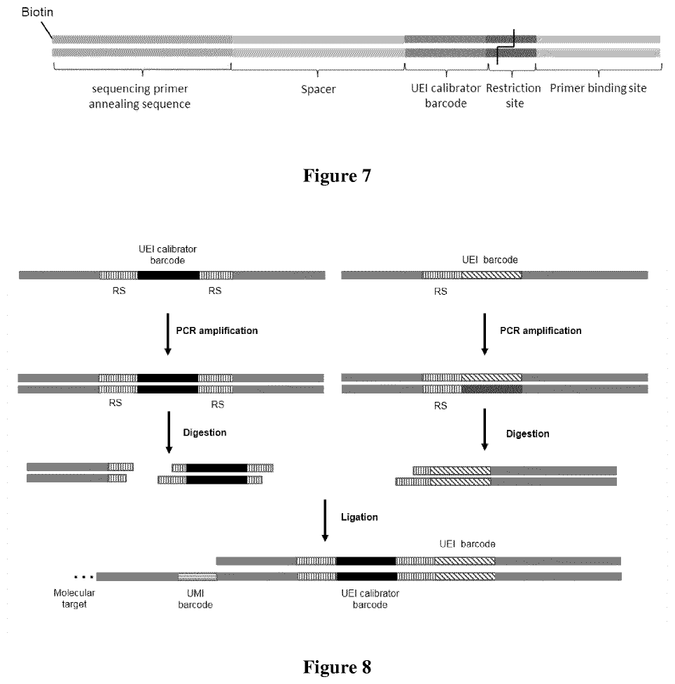

Optionally, UEI-calibrator sequences may further comprise an additional region comprised between the UEI-calibrator barcode and the sequencing primer annealing sequence when present. This region acts as a spacer between the sequencing primer annealing sequence and the UEI-calibrator barcode and may be used to adjust the length of the amplified sequences. An exemplary illustration of such UEI-calibrator sequence is presented in FIG. 7.

In another embodiment, UEI-calibrator sequences comprise two overhang producing restriction sites which generate, upon digestion with the corresponding restriction enzyme, on one side an overhang compatible with overhangs of digested UEI sequences comprising UEI barcodes and on the other side an overhang compatible with overhangs of digested UMI sequences comprising UMI barcodes.

In this embodiment, UEI-calibrators may comprise constant regions adjacent to each of the two overhang producing restriction sites. These constant regions allow amplifying UEI-calibrator sequences, preferably using PCR amplification. In particular, these regions may comprise primer binding sites. Preferably, these constant regions have a length of 10 to 35 nucleotides, preferably of 15 to 25 nucleotides. Preferably these constant regions are orthogonal to that of UEI and UMI sequences in order to prevent unwilling hybridization.

Preferably, all UEI-calibrator sequences have to same sequence except for the UEI-calibrator barcode, i.e. they exhibit, the same restriction site, the same constant region(s) and optionally the same sequencing primer annealing sequence.

Encapsulation, Amplification and Assembling of the Identification Sequence

In order to obtain droplets of the second set,

a plurality of UEI sequences, UEI-calibrator sequences and UMI molecules are encapsulated with an amplification reaction mixture within emulsion droplets,

UEI sequences and UEI-calibrator sequences are amplified within droplets; and

UEI-calibrator barcodes, UEI barcodes and UMI molecules are assembled through restriction enzyme digestion and ligation of compatible overhangs.

Each droplet comprises one or several UEI sequences, one or several UEI-calibrator sequences and a plurality of unique UMI molecules.

The combination of UEI sequences and UEI-calibrator sequences is different for each droplet and each droplet comprising a plurality of unique UMI molecules.

This encapsulation can be carried out by any routine method known by the skilled person.

The dilution of each solution comprising UEI sequences, UEI-calibrator sequences and UMI molecule can be easily adjusted in order to control droplet occupancy and molecule distribution according to Poisson statistics.

UEI sequences and UEI-calibrator sequences may be encapsulated in single or double stranded form, preferably in double stranded form.

Multiplex amplification of UEI sequences and UEI-calibrator sequences within droplets may be performed using any method known by the skilled person.

The amplification reaction mixture comprises all reagents required to perform DNA amplification into the droplets, i.e. typically a DNA polymerase, primers, buffers, dNTPs, salts (e.g. MgCl2), etc. . . . . Preferably, primers are designed in order to allow the complete amplification of the sequences. In certain embodiments, amplification relies on alternating cycles of heating and cooling (i.e., thermal cycling) to achieve successive rounds of replication (e.g., PCR). Methods of amplifying genetic elements compartmentalized in emulsion droplets are well-know and widely practiced by the skilled person (see for example, Chang et al. Lab Chip. 2013 Apr. 7; 13(7):1225-42; Zanoli and Spoto, Biosensors 2013, 3, 18-43). In particular, the amplification may be performed by any known technique such as polymerase chain reaction (PCR), nucleic acid sequence-based amplification (NASBA), loop-mediated isothermal amplification (LAMP), helicase-dependent amplification (HDA), rolling circle amplification (RCA), multiple displacement amplification (MDA) and recombinase polymerase amplification (RPA). The suitable method can be easily chosen by the skilled person depending on the nature of the encapsulated genetic elements.

Preferably, UEI sequences and UEI-calibrator sequences are amplified into the droplets by multiplex PCR amplification.

Optionally, UEI sequence and UEI-calibrator sequences may be assembled through their compatible overhangs before amplification. In this case, amplification reaction directly amplifies a fragment comprising UEI sequence and UEI-calibrator sequence. UMI molecules and amplification products are then assembled through restriction enzyme digestion and ligation of compatible overhangs.

After encapsulation and amplification of UEI and UEI-calibrator sequences, identification sequences comprising UMI, UEI and UEI-calibrator barcodes are assembled through restriction enzyme digestion and ligation of compatible overhangs.

In some embodiments, identification sequences comprise a DNA moiety of UMI molecule ligated to a UEI-calibrator sequence and a UEI-calibrator sequence ligated to a UEI sequence. An exemplary illustration of such embodiment is presented in FIG. 8.

Alternatively, an identification sequence may comprise a DNA moiety of UMI molecule ligated to a UEI sequence and a UEI sequence ligated to a UEI-calibrator sequence.

Restriction sites comprised on UMI, UEI and UEI calibrator sequences may be recognized by the same enzyme or by different enzymes.

Preferably, the enzyme recognizing the restriction site allowing assembling of UEI and UEI calibrator sequences does not recognize the restriction site of the UMI molecule. UEI sequence and UEI-calibrator sequences may thus be assembled through their compatible overhangs before amplification and then attached to the UMI molecule.

Preferably, restrictions sites generating the overhangs are chosen in order to ensure that a productive ligation event leads to the destruction of the restriction site. Therefore, the resulting chimeric molecule will not be a substrate of the restriction enzymes present in the mixture and the equilibrium is pulled toward the formation of the wished ligation products.

Preferably, the restriction sites generating the overhangs are non-palindromic in order to ensure directionality of the association of UMI, UEI and UEI calibrator sequences.

Restriction enzymes, DNA ligase and optionally buffer may be provided in the aqueous phase during the encapsulation step (i.e. with the amplification mixture) or may be added subsequently e.g. by pico-injection or droplet fusion.

The emulsion may be then collected and incubated to allow digestion and ligation and thus association of identification sequences.

Thus, after ligation, the droplets of the second set comprise probes, wherein each probe comprises (i) a capture moiety which will specifically detect molecular targets contained in droplets of the first set and (ii) an identification sequence comprising a combination of UMI, UEI and UEI-calibrator barcodes. This combination constitutes a signature unique allowing identifying each target molecule and reassigning it to its original entity.

Droplet Fusion and Labelling of Molecular Targets

As described above, the first set of emulsion droplets comprises molecular targets and the second set of emulsion droplets comprises probes comprising a capture moiety capable of specific binding or ligation to a molecular target contained in droplets of the first set or to an adaptor linked to said molecular target, and a DNA moiety comprising an identification sequence (i.e. a sequence comprising a unique combination of UMI, UEI and UEI-calibrator barcodes).

The method of the invention comprises fusing droplets of the first set with droplets of the second set wherein a droplet of the first set is fused with no more than one droplet of the second set.

Any technique known by the skilled person may be used to fuse a first droplet and a second droplet together to create a combined droplet. For example, opposite electric charges may be given to the first and second droplets (i.e., positive and negative charges, not necessarily of the same magnitude), which may increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc.

Any technique known by the skilled person may be used to ensure that a droplet of the first set is fused with no more than one droplet of the second set. In particular, droplets may be paired through a pairing channel before to reach the coalescence point. The use of such channel is well-known by the skilled person in order to control fusion of microfluidic droplets. In embodiments wherein a pairing channel is used, droplets of the first and second sets should have different sizes. The pairing channel is a long channel having a width larger than the smallest droplets and narrower than the largest droplets. As a consequence, the small droplet catches the large one and pairs of droplets are formed at the exit of the channel. This configuration ensures that droplets properly pairwise prior to reaching the coalescence point.

In some embodiments, droplets of the first set and/or droplets of the second sets are generated on separate microfluidic system(s) and re-injected into the device. Preferably, droplets are spaced with oil streams and synchronized before to enter the pairing channel.

Preferably at least 60%, more preferably at least 80% of droplets, and even more preferably at least 95% of the first set are fused with a droplet of the second set.

After droplet fusion, molecular targets contained in the droplets of the first set are labelled with probes provided in the droplets of the second set.

This labelling can occur spontaneously, in particular when the probe is a chimeric probe, or may require an additional step, in particular when the molecular target is a nucleic acid.

Nucleic acid molecular targets may be labelled using probes as described above and comprising a capture moiety which is specific to a particular DNA or RNA molecule or to an adaptor, as priming sites for a DNA polymerase synthetizing complementary strands of molecular targets. DNA and/or RNA molecular targets are converted into barcoded complementary DNA (cDNA) upon reverse transcription or other DNA polymerization reaction.

In some embodiments, some molecular targets are RNA molecules and the DNA polymerase is a reverse transcriptase. Upon hybridization of the probe, through its capture moiety, to the targeted RNA or the adaptor, reverse transcription can occur and first strand of complementary DNA (cDNA) can be synthesized.

It will be recognized that DNA polymerization reaction or reverse transcription requires appropriate conditions, for example the presence of an appropriate buffer and DNA polymerase enzyme, temperatures appropriate for annealing of the probes to targeted RNAs or DNAs and the activity of the enzyme and optionally presence of DTT. These conditions mainly depend on the polymerase and may be adapted according to the supplier guidance.

Additional reagents required for the labelling may be provided in droplets of the first or second set, in particular may be added to the aqueous phase of the droplets before fusion of the two sets, (e.g. before encapsulation i.e. by direct inclusion in the mixture or via a co-flow or after droplet generation by any known technique such as pico-injection or droplet fusion) or after the fusion of the two sets by any known technique such as pico-injection or droplet fusion.

In a particular embodiment wherein the molecular target is a nucleic acid, preferably a RNA, a sequencing primer annealing sequence may be added to labelled molecular target after incorporating identification sequence. In particular, the method may further comprise, after incorporating identification sequence, performing primer extension reaction using primers comprising from their 3′ end to their 5′ end, a region that hybridizes to complementary strands of molecular targets, i.e. to cDNA, and a sequencing primer annealing sequence.

Optionally, at their 5′ extremity, the primers may further comprise a binding tag allowing the specific capture of primer extension reaction products. Preferably, the binding tag is biotin or digoxigenin, more preferably is biotin.

Primer extension reaction may be performed as described above and reaction mixture may be brought into the droplet using any known method such as pico-injection or droplet fusion. Alternatively, the primer extension reaction can be performed in bulk upon droplet breaking and content recovery.

After completion of the labelling process, droplets may be broken and their content may be recovered, i.e. droplet lysate, e.g. to be further analysed/sequenced.

Analysis of Molecular Targets Labelled with the Method of the Invention

In a further aspect, the present invention also relates to a method of quantifying one or several molecular targets from a plurality of entities with single-entity resolution, said method comprising

labelling said molecular targets according to the method of the invention as described above, capturing said labelled molecular targets comprising identification sequences (i.e. UMI, UEI and UEI-calibrator barcodes),

amplifying identification sequences

sequencing said amplified sequences.

All embodiments described above for the method of the invention are also encompassed in this aspect.

The step of capturing labelled molecular targets comprising identification sequences allows removing from the droplet lysate untargeted molecules and unreacted probes. This step may be performed using any capture molecule which is able to specifically bind molecular targets such as an antibody or nucleic acid specific of a molecular target, attached to a support. The capture molecule may be directly or indirectly attached to the support. In particular, the capture molecule may comprise a binding tag, e.g. biotin, interacting with a partner, e.g. streptavidin, linked to the support. The capture molecule may be for example a biotinylated monoclonal antibody specific of a targeted protein, or synthetic DNA oligonucleotide specific of a cDNA produced from a targeted RNA. A binding tag can be added during the synthesis of the second cDNA strand by primer extension mentioned above. The support may be chosen in order to allow washing of unreacted molecules. Preferably the support is beads, more preferably streptavidin conjugated beads. In some preferred embodiments, the support is magnetic beads, in particular streptavidin conjugated magnetic beads.

The identification sequences are then sequenced using any method known by the skilled person, preferably using a next generation sequencing method.

In some embodiments, wherein said identification sequences do not comprise any sequencing primer annealing sequences, said sequences may be added before the sequencing step, preferably by DNA amplification using primers comprising said sequences or by ligation of oligonucleotides comprising said sequences.

Preferably, when the molecular target is RNA, a part of its cDNA is also sequenced.

One of the main advantage of the present invention is the possibility of pooling all captured labelled molecular targets prior to the sequencing step, whatever the type of molecular targets (e.g. RNA or protein). Sequences may be then analyzed using any method known by the skilled person such as bioinformatics to cluster said sequences and determining the absolute quantification of molecular targets. Firstly, UEI and UEI-calibrator barcode combinations are used to cluster molecules according to their entity of origin. Secondly, molecules are clustered according to their type and identity and redundant sequences are eliminated using the UMI barcodes, giving access to the absolute quantification of each molecule with single-entity resolution.

Microfluidic Devices

In a further aspect, the present invention also relates to a microfluidic device suitable for implementing at least one step of the methods of the invention.

All embodiments described above for the methods of the invention are also encompassed in this aspect.

As used herein, the term “microfluidic device”, “microfluidic chip” or “microfluidic system” refers to a device, apparatus or system including at least one microfluidic channel.

The microfluidic system may be or comprise silicon-based chips and may be fabricated using a variety of techniques, including, but not limited to, hot embossing, molding of elastomers, injection molding, LIGA, soft lithography, silicon fabrication and related thin film processing techniques. Suitable materials for fabricating a microfluidic device include, but are not limited to, cyclic olefin copolymer (COC), polycarbonate, poly(dimethylsiloxane) (PDMS), poly(methyl methacrylate) (PMMA), and glass. Preferably, microfluidic devices are prepared by standard soft lithography techniques in PDMS and subsequent bonding to glass microscope slides. Due to the hydrophilic or hydrophobic nature of some materials, such as glass, which adsorbs some proteins and may inhibit certain biological processes, a passivating agent may be necessary. Suitable passivating agents are known in the art and include, but are not limited to silanes, fluorosilanes, parylene, n-dodecyl-β-D-maltoside (DDM), poloxamers such as Pluronics.

As used herein, the term “channel” refers to a feature on or in an article (e.g., a substrate) that at least partially directs the flow of a fluid. The term “microfluidic channel” refers to a channel having a cross-sectional dimension of less than 1 mm, typically less than 500 μm, 200 μm, 150 μm, 100 μm or 50 μm, and a ratio of length to largest cross-sectional dimension of at least 2:1, more typically at least 3:1, 5:1, 10:1 or more. It should be noted that the terms “microfluidic channel”, microchannel” and “channel” are used interchangeably in this description. The channel can have any cross-sectional shape (circular, oval, triangular, irregular, square or rectangular, or the like). Preferably, the channel has a square or rectangular cross-sectional shape. The channel can be, partially or entirely, covered or uncovered. As used herein, the term “cross-sectional dimension” of a channel is measured perpendicular to the direction of fluid flow.

The microfluidic device of the invention comprises
a first emulsion re-injection module or on-chip droplet generation module;
a second emulsion re-injection module or on-chip droplet generation module
a droplet-pairing module, and
optionally a module coupling droplet fusion to injection,

wherein emulsion re-injection modules and/or on-chip droplet generation modules are in fluid communication and upstream to the droplet-pairing module, the droplet-pairing module is in fluid communication and upstream to the module coupling droplet fusion to injection.

As used herein, the term “upstream” refers to components or modules in the direction opposite to the flow of fluids from a given reference point in a microfluidic system.

As used herein, the term “downstream” refers to components or modules in the direction of the flow of fluids from a given reference point in a microfluidic system.

In an embodiment, the microfluidic device of the invention comprises

two emulsion re-injection modules

a droplet-pairing module, and

optionally a module coupling droplet fusion to injection.

In another embodiment, the microfluidic device of the invention comprises

an emulsion re-injection module and an on-chip droplet generation module,

a droplet-pairing module, and

optionally a module coupling droplet fusion to injection.

In a further embodiment, the microfluidic device of the invention comprises

two on-chip droplet generation modules,

a droplet-pairing module, and

optionally a module coupling droplet fusion to injection.

The emulsion re-injection module may be easily designed by the skilled person based on any known techniques. Typically, an emulsion re-injection module comprises a v-shaped structure where injected droplets are spaced by carrier oil supplying by at least one, preferably two side channels connected with the re-injection channel.

The module for generating droplets may be easily designed by the skilled person based on any known techniques. For example, emulsion droplets may be produced in the droplet generation module by any technique known by the skilled person such as drop-breakoff in co-flowing streams, cross-flowing streams in a T-shaped junction (see for example WO 2002/068104), and hydrodynamic flow-focusing (reviewed by Christopher and Anna, 2007, J. Phys. D: Appl. Phys. 40, R319-R336).

As explained above, the droplet-pairing module is a channel with dimensions allowing the contact between droplets of the two sets. In an embodiment, the width of the channel is about the diameter of the larger droplets and the depth of the channel is lower than the diameter of the larger droplets. In another embodiment, the depth of the channel is about the diameter of the larger droplets and the width of the channel is lower than the diameter of the larger droplets.

The length of the pairing channel has to be sufficient to obtain a contact between droplets of the first and second sets. Preferably, the time of contact is greater than 1 ms, preferably greater than 4 ms. As used herein, “the contact time t” refers to the time in which paired droplets stay in physical contact before reaching the end of the pairing channel. Typically, the length of the pairing channel is ranging from 100 μm to 10 mm, preferably from 500 μm to 2 mm, and more preferably is about 1.5 mm.

The module coupling droplet fusion to injection is preferably a module wherein droplets, after pairing, are exposed to an electric field destabilizing their interface thanks to the proximity of electrodes, and are, in the same time, contacted with a stream injected in the channel. Destabilization of the interface then leads not only to droplet coalescence but also to infusion of the injected stream into droplets.

The microfluidic device may further comprise a collection module wherein fused droplets are recovered.

Optionally, the microfluidic device of the invention may comprise an inlet downstream to the module coupling droplet fusion to injection and upstream to the collection module. This inlet may be used to inject additional surfactant in order to increase the stability of fused droplets and to prevent any coalescence during the storage.

In another aspect, the present invention also relates to a kit comprising one or several microfluidic devices according to the invention and as described above.

The kit may further comprise one or several microfluidic chips comprising an on-chip droplet generation module.

The kit of the invention may further comprise
one or several probes as described above; and/or
one or several UMI molecules as described above; and/or
one or several UEI sequences as described above; and/or
one or several UEI-calibrator sequences as described above; and/or
one or several primers suitable to amplify UEI sequences and/or UEI-calibrator sequences; and

optionally
an aqueous phase and/or an oil phase; and/or
a leaflet providing guidelines to use such a kit.

All embodiments described above for the methods and the microfluidic device of the invention are also encompassed in this aspect.

The present invention further relates to the use of a kit of the invention to label a plurality of molecular targets from a plurality of entities according to the method of the invention, or to quantify one or several molecular targets from a plurality of entities according to the method of the invention. All embodiments described above for the methods, the microfluidic device and the kit of the invention are also encompassed in this aspect.

As used herein, the verb “to comprise” is used in its non-limiting sense to mean that items following the word are included, but items not specifically mentioned are not excluded.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

As used herein, the term “about” refers to a range of values±10% of the specified value. For example, “about 20” includes ±10% of 20, or from 18 to 22. Preferably, the term “about” refers to a range of values±5% of the specified value.

All patent and literature references cited in the present specification are hereby incorporated by reference in their entirety.

### EXAMPLES

I. Microfluidics Chips Fabrication and Operation

In all the examples presented below microfluidic chips were fabricated using the same procedure and they were manipulated using on the same workstation.

a. Micro Fluidic Chips Preparation and Operation

Microfluidic devices were obtained using a classic replica molding process as described previously in (Mazutis et al., 2009). Briefly, devices were designed on Autocad (Autodesk 2014), negative photomasks were printed (Selba S.A.) and used to prepare molds by standard photolithography methods. SU8-2010 and SU8-2025 photoresist (MicroChem Corp.) were used to pattern 10 to 40 μm deep channels onto silicon wafers (Siltronix). Microfluidic devices were then fabricated in polydimethylsiloxane (PDMS, Silgard 184, Dow-Corning) using conventional soft lithography methods (Xia and Whitesides, 1998). Upon plasma bonding to a glass slide, channels were passivated with a solution of 1% (v/v) 1H, 1H, 2H, 2H-perfluorodecyltrichlorosilane (97%, ABCR GmbH and Co,) in HFE7500 (3M) and subsequently flushed with compressed air. Key dimensions and depth of microfluidic devices are given on concerned figures and in their captions.

Aqueous phases were loaded in I.D. 0.75 mm PTFE tubings (Thermo Scientific) and oils were loaded in 2 mL Micrew Tubes (Thermo Scientific). Liquids were injected into microfluidic devices at constant and highly controlled flow-rates using a 7-bar MFCS™ pressure-driven flow controller (Fluigent) equipped with Flowells (7 μL/min flow-meters) allowing for operation in flow-rate controlled mode.

b. Optical Set-Up, Data Acquisition and Control System

The optical setup was based on an inverted microscope (Nikon Eclipse Ti-S) mounted on a vibration-dampening platform (Thorlabs B75150AE). The beams of a 488 nm laser (CrystaLaser DL488-050-O) and a 561 nm laser (Cobolt DPL 561-NM-100MW) were combined using a dichroic mirror (Semrock 2F495-DI03-2536). They were further combined with a 375 nm laser (CrystaLaser DL375-020-O) using a second dichroic mirror (Semrock Di02-R405-25x36) prior to shaping the resulting combined beam as lines using a pair of lenses (Semrock LJ1878L2-A and LJ1567L1-A) that was directed into the microscope objective (Nikon Super Plan Fluor 20×ELWD or Nikon Super Plan Fluor 40×ELWD) to be focused in the middle of the channel at the detection point. The emitted fluorescence was collected by the same objective and separated from the laser beams by a multi-edges dichroic mirror (Semrock Di01-R405/488/561/635-25x36). Blue (7-aminocoumarin-4-methanesulfonic acid) fluorescence was resolved from green (Syto9, FAM) and orange (Texas-Red) fluorescence by a third dichroic mirror (Semrock LM01-480-25). Then green fluorescence was separated from orange fluorescence by an additional dichroic mirror (Semrock FF562-Di03-25x36). Fluorescence was finally measured by three photomultiplier tubes (Hamamatsu H10722-20) equipped with bandpass filters (Semrock FF01-445/45-25, FF01-600/37-25 and FF03-525/50-25 for blue, green and orange detection respectively). Signal acquisition from the PMTs was performed using an intelligent data acquisition (DAQ) module featuring a user-programmable FPGA chip (National Instruments PCI-7851R) driven by internally developed firmware and software. To monitor the experiment, we used an additional dichroic mirror (Semrock FF665-Di02-25x36) to split light to a CCD camera (Allied Vision Technologies Guppy F-033). A long-pass filter (Semrock BLP01-664R-25) prevented potentially damaging reflections of the lasers into the camera.

II. Sequences Used in the Examples

TABLE 1
Sequences used in the examples

| | SEQ | |
|---|---|---|
| Molecule | ID no | 5′- Sequence - 3′ |
| Template | 1 | GAGCGGATAACAATTTCACACAGGCACGGGGTGTGAGATCACAGA |
| UEI-DraIII | | TCGGAAGAGCGTCGTGT |
| UEI-Fwd | 2 | GAGCGGATAACAATTTCACACAGG |
| UEI-Rev | 3 | ACACGACGCTCTTCCGATC |
| Template Calibrator- | 4 | TCGTCGGCAGCGTCAGATGTGTATAAGAGACAGGGAAGACGTAGCA |
| N15-AlwNI | | AGGGAGTAGCCAATGTGAATTGAGAGCCTTAAGCTGTATNNNNNNN |
| | | NNNNNNNNNNNNNNNNNNCAGGGGCTGGTCGTGACTGGGAAAAC |
| | | CCTGGC |
| Calib-Rev | 5 | GCCAGGGTTTTCCCAGTCACGAC |
| Illu-2-Fwd | 6 | TCGTCGGCAGCGTCAGATG |
| Template UEI-N15- | 7 | GAGCGGATAACAATTTCACACAGGCACGGGGTGNNNNNNNNNNNN |
| DraIII* | | NNNGATCGGAAGAGCGTCGTGT |
| Template Calibrator- | 8 | TCGTCGGCAGCGTCAGATGTGTATAAGAGACAGGGAAGACGTAGCA |
| N15-AlwNI-1g | | AGGGAGTAGCCAATGTGAATTGAGAGCCTTAAGCTGTATCAGGACC |
| | | AGAGAGATGANNNNNNNNNNNNNNNCAGGGGCTGGTCGTGACTG |
| | | GGAAAACCCTGGC |
| Illu1 rev UEI | 9 | GTCTCGTGGGCTCGGAGATGTGTATAAGAGACAGACACGACGCTCT |
| | | TCCGATC |
| Illu1 | 10 | GTCTCGTGGGCTCGGAGAT |
| Template-AlwNI- | 11 | GGAAGACGTAGCAAGGGAGTAGCCAATGTGAATTCACACAGTGNN |
| N15-Calib-DraIII | | NNNNNNNNNNNNNCAGGGGCTGGTCGTGACTGGGAAAACCCTGGC |
| Calib-Fwd | 12 | GGAAGACGTAGCAAGGGAGTAGCC |
| RTmimicks | 13 | [phosphate]CTGCGCGGATCCCGGAAGCGAGGCCAGCTGGCTGCNNNN |
| | | NNNNTTGGCTGTCTCTTATACACATCTGACGCTGCCGACGA |
| antiSBACA-AlwN1 | 14 | GCAGCCAGCTGGCCTCGCTTCCGGGATCCGCGCAGACA |
| Template UEI-N15- | 15 | GAGCGGATAACAATTTCACACAGGCACGGGGTGNNNNNNNNNNNN |
| DraIII-Illu1 | | NNNGATCGGAAGAGCGTCGTGTCTGTCTCTTATACACATCTCCGAGC |
| | | CCACGAGAC |
| RNA III | 16 | AUUAAUACGACUCACUAUACCUAGAUCACAGAGAUGUGAUGGAAA |
| | | AUAGUUGAUGAGUUGUUUAAUUUUAAGAAUUUUUAUCUUAAUUA |
| | | AGGAAGGAGUGAUUUCAAUGGCACAAGAUAUCAUUUCAACAAUC |
| | | GGUGACUUAGUAAAAUGGAUUAUCGACACAGUGAACAAAUUCAC |
| | | UAAAAAAUAAGAUGAAUAAUUAAUUACUUUCAUUGUAAAUUUGU |
| | | UAUCUACGUAUAGUACUAAAAGUAUGAGUUAUUAAGCCAUCCCAA |
| | | CUUAAUAACCAUGUAAAAUUAGCAAGUGAGUAACAUUUGCUAGU |
| | | AGAGUUAGUUUCCUUGGACUCAGUGCUAUGUAUUUUUCUUAAUU |
| | | AUCAUUACAGAUAAUUAUUUCUAGCAUGUAAGCUAUCGUAAACA |
| | | ACAUCGAUUUAUCAUUAUUUGAUAAAUAAAAUUUUUUUCAUAAU |
| | | UAAUAACAUCCCCAAAAAUAGAUUGAAAAAAUAACUGUAAAACA |
| | | UUCCCUUAAUAAUAAGUAUGGUCGUGAGCCCCUCCCAAGCUCGCG |
| | | GCCUUUUG |
| RT-UMI-RNAIII* | 18 | [phosphate]CTGCGCGGATCCCGGAAGCGAGGCCAGCTGGCTGCNNNN |
| | | NNTTGGGAGGGGCTCA |
| SB-alone | 20 | CTGCGCGGATCCCGGAAGCGAGGCCAGCTGGCTGC |
| RNAIII-Fwd | 21 | TCGTCGGCAGCGTCAGATGTGTATAAGAGACAGATAACTGTAAAAC |
| | | ATTCCCTTAATAATAAG |
| Gfp mRNA | 22 | AUGACCAGCUACCCAUACGAUGUUCCAGAUUACGCUAUCGAAGGC |
| | | CGCGGCCGCCAUCAUCAUCAUCAUCAUGAUAUCGGUACCAGUAAA |
| | | GGAGAAGAACUUUUCACUGGAGUUGUCCCAAUUCUUGUUGAAUU |
| | | AGAUGGUGAUGUUAAUGGGCACAAAUUUUCUGUCAGUGGAGAGG |
| | | GUGAAGGUGAUGCAACAUACGGAAAACUUACCCUUAAAUUUAUU |
| | | UGCACUACUGGAAAACUACCUGUUCCAUGGCCAACACUUGUCACU |
| | | ACUUUCGCGUAUGGUCUUCAAUGCUUUGCGAGAUACCCAGAUCAU |
| | | AUGAAACAGCAUGACUUUUUCAAGAGUGCCAUGCCCGAAGGUUAU |
| | | GUACAGGAAAGAACUAUAUUUUUCAAAGAUGACGGGAACUACAA |
| | | GACACGUGCUGAAGUCAAGUUUGAAGGUGAUACCCUUGUUAAUA |
| | | GAAUCGAGUUAAAAGGUAUUGAUUUUAAAGAAGAUGGAAACAUU |
| | | CUUGGACACAAAUUGGAAUACAACUAUAACUCACACAAUGUAUAC |
| | | AUCAUGGCAGACAAACAAAAGAAUGGAAUCAAAGUUAACUUCAA |
| | | AAUUAGACACAACAUUGAAGAUGGAAGCGUUCAACUAGCAGACCA |
| | | UUAUCAACAAAAUACUCCAAUUGGCGAUGGCCCUGUCCUUUUACC |
| | | AGACAACCAUUACCUGUCCACACAAUCUGCCCUUUCGAAAGAUCC |
| | | CAACGAAAAGAGAGACCACAUGGUCCUUCUUGAGUUUGUAACAGC |
| | | UGCUGGGAUUACACAUGGCAUGGAUGAACUAUACAAAGAGAAUU |
| | | CAGAGCUCGGAUCCACUCGAGAUGCAUUAGAACAAAAAUUAUUAU |
| | | CAGAAGAAGAUUUAAAUUAA |
| gfp-mut2-RT-UMI- | 23 | [phosphate]CTGCGCGGATCCCGGAAGCGAGGCCAGCTGGCTGCNNNN |
| AlwNI* | | NNNNCAAATAAATTTAAGGGTAAGTTTTCC |
| Gfp-Fwd-25 nt | 24 | TGAATTAGATGGTGATGTTAATGGGC |
| Template-UEI- | 25 | GAGCGGATAACAATTTCACACAGGCACGGGGTGNNACNNGANNCT |
| DraIII-N2x5 | | NNGCNNGATCGGAAGAGCGTCGTGT |
| Template-UEI- | 26 | GAGCGGATAACAATTTCACACAGGCACGGGGTGNNNNACANNNNG |
| DraIII-N4443 | | AGNNNNTCTNNNGATCGGAAGAGCGTCGTGT |
| Template-Calibrator- | 27 | GGAAGACGTAGCAAGGGAGTAGCCAATGTGAATTCACACAGTGTC |
| AlwNI | | TACAAGTACAGGGGCTGGTCGTGACTGGGAAAACCCTGGC |
| Template-Calibrator- | 28 | GGAAGACGTAGCAAGGGAGTAGCCAATGTGAATTCACACAGTGNN |
| AlwNI-N2x5 | | TCNNTANNCANNAGNNCAGGGGCTGGTCGTGACTGGGAAAACCCT |
| | | GGC |
| Template-Calibrator- | 29 | GGAAGACGTAGCAAGGGAGTAGCCAATGTGAATTCACACAGTGNN |
| AlwNI-N4443 | | NNTCTNNNNACANNNNGAGNNNCAGGGGCTGGTCGTGACTGGGAA |
| | | AACCCTGGC |
| Template-Calibrator- | 30 | GGAAGACGTAGCAAGGGAGTAGCCAATGTGAATTCACACAGTGNN |
| AlwNI-N15 | | NNNNNNNNNNNNNCAGGGGCTGGTCGTGACTGGGAAAACCCTGGC |
| Tag-NaBAb-AlwNI | 31 | CACACAGGAAACAGCTATGACCCAGTGTCTGNNNNNNNNTGTGGA |
| | | CGCTGTCTCTTATACACATCTGACGCTGCCGACGACGTCGTGACTGG |
| | | GAAAACCC |
| BG-M13-Fwd** | 32 | [BG]-isp18-GGGTTTTCCCAGTCACGACG* |
| Alexa488-M13-Rev | 33 | [Alexa488]-CACACAGGAAACAGCTATGACC |
| Template UEI-N15- | 36 | GAGCGGATAACAATTTCACACAGGCACGGGGTGNNNNNNNNNNNN |
| DraIII-Illu2 | | NNNAACTGTCTCTTATACACATCTGACGCTGCCGACGA |
| RNAIII-Fw-89-Lg | 37 | GTCTCGTGGGCTCGGAGATGTGTATAAGAGACAGTCATAATTAATA |
| | | ACATCCCCAAAAATAGATTG |

All the oligonucleotides (but 15 obtained by primer extension, 16 and 22) were purchased from Integrated DNA Technologies (IDT). Random sequences corresponding to the UCI or the Calibrator are represented by N arrays and are underlined whereas the UMI is represented by an italicized N arrays. The sequence digested by AlwNI (CAGNNNCTG) and DraIII (CACNNNGTG) are shown in bold.
*These oligonucleotides are 5′phosphorylated to allow their ligation with another DNA by T4 DNA ligase.
**[BG]: benzyl-guanine; isp18: 18 carbon-long internal spacer.

### Example 1: Cells Preparation, Individualization and Lysis

In this example, we demonstrated that bacterial cells can be individualized in droplets and efficiently lysed using a detergent. E. coli bacteria (T7-Xpress Lys/I^{q}, GFP null strain New England Biolabs) transformed either with a plasmid carrying GFP gene under the control of T7 RNA polymerase promoter or a plasmid bearing an unrelated construct were used as model bacteria and will be later summarized as strains GFP+ and GFP− respectively. Note that both plasmids confer ampicillin resistance to the bacteria allowing for their co-culture.

a. Preparation of the Cell

A cell pre-culture (starter) was first obtained by inoculating a 2YT media supplemented with 0.1 mg/mL ampicillin and 2% glucose, and incubating it over-night at 37° C. and under agitation. 500 μL of this starter (OD₆₀₀ of 3.77) were then used to inoculate 20 mL of the same medium, and the bacteria were allowed to grow until the culture reached an OD₆₀₀ of 0.6. Next, the cells were fluorescently stained to allow for monitoring cells during the microfluidic steps. To do so, 2 mL of this culture were stained using 5 μM SYTO 9 (molecular probes by Life technologies, ref 34854) for 2 hours in the dark, following supplier recommendations. Cells were washed once with PBS buffer 1× before being pelleted for further experiments.

b. Cells Encapsulation and Lysis

Experimental Procedure

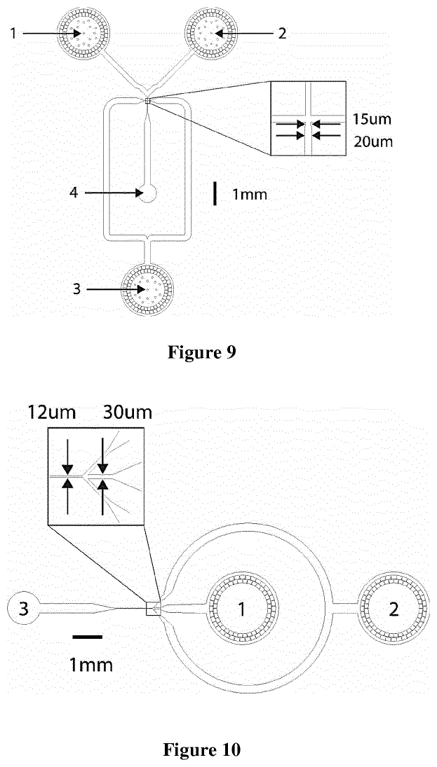

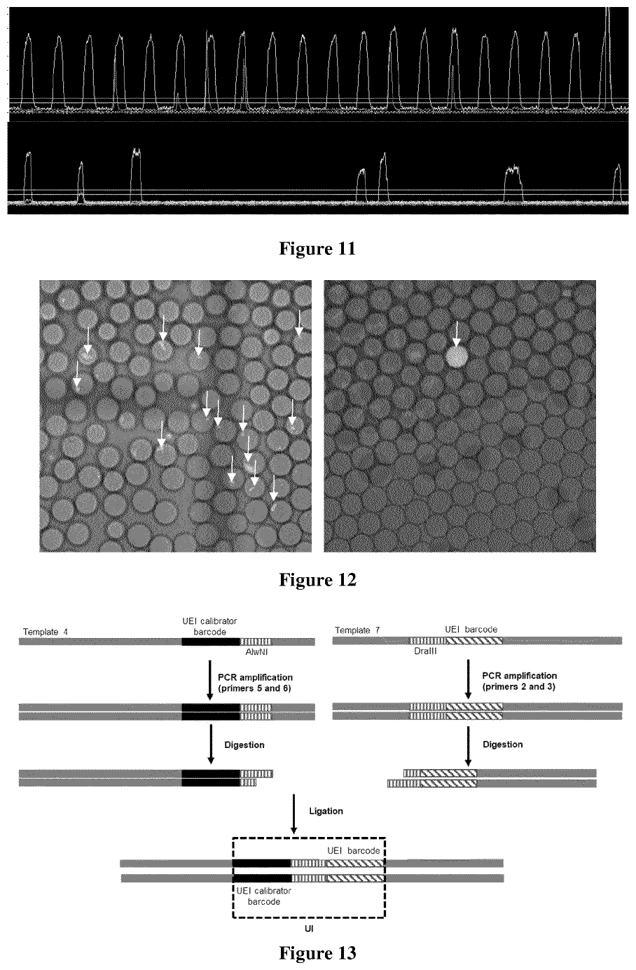

Pellets of labelled cells were diluted to reach an OD₆₀₀ of 3.75 (giving a droplet occupancy of ˜20%) in a CutSmart® buffer 1× (50 mM Potassium acetate, 20 mM Tris acetate pH 7.9 at 25° C., 10 mM Magnesium acetate and 0.1 mg/mL BSA, New England Biolabs) supplemented with 37.5 μg/mL dextran Texas red 70 000 MW (used as droplet tracker), and 0.05% Pluronic F68. The mixture was then loaded into a 0.5 mL PCR tube containing a magnetic stirring bar (5 mm length and 2 mm diameter) closed by a plug of PDMS. One extremity of the system was connected to a Fluigent infusion device whereas the other extremity was connected to the droplet co-flow generator (FIG. 9, inlet 1). During droplet production, the mixture was stirred by placing a stirring plate (Hei-Mix S, Heidolph instrument) aside the bacteria-containing tube to keep bacteria properly suspended. A solution of CutSmart® buffer 1× supplemented with B-PER lysis reagent (90% final concentration Thermo Scientific) was loaded into a length of PTFE tubing (I.D. 0.75 mm tubing; Thermo Scientific) and connected to the Fluigent infusion device at one side of the tubing and to the droplet co-flow generator (FIG. 9, inlet 2) at the other side of the tubing. Both aqueous solutions (bacteria suspension and lysis solution) were combined on-chip prior to being dispersed into a stream of Novec 7500 (3M) fluorinated oil supplemented with 3% Krytox-Jeffamine 1000 diblock copolymer fluorosurfactant (Ryckelynck et al 2015) that was infused into the third inlet (FIG. 9, inlet 3). 4 pL droplets were then produced at a rate of 800 droplets per second by infusing both aqueous phases at 200 nL/min and the oil phase at 1000 nL/min. Droplets were collected at the outlet of the chip (FIG. 9, outlet 4) via a length of tubing into a 0.2 mL PCR tube closed by a plug of PDMS. Moreover, an identical experiment in which the lysis solution was exchanged for a lysis agent-free CutSmart® buffer 1× phase was used for a control experiment. Upon an incubation of 20 min at 25° C., droplet fluorescence was analyzed on-line by re-injecting the droplets into a droplet fluorescence analysis microfluidic device. Droplets contained into a 0.2 mL PCR tube closed by a plug of PDMS were reinjected into a droplet analyzer (FIG. 10, inlet 1) where they were spaced by an oil stream (FIG. 10, inlet 2) and their fluorescence content was analyzed by the optical set-up introduced above. The Texas Red contained into each droplet allows identifying a droplet as an orange peak (FIG. 11). Moreover, the staining of cell nucleic acids by Syto9 enables both detecting bacteria and appreciating their lysed/integer status. Indeed, the presence of an integer cell is indicated by a spike of green fluorescence into the droplet (orange peak; FIG. 11, top panel), whereas a lysed cell gives a more homogeneous labelling of lower intensity (e.g. second droplet from the left on FIG. 11, bottom panel). To further confirm this result, 10 μL of the droplets were loaded into a plastic Malassez hematimeter and imaged on an epi-fluorescence microscope both in bright-field and green fluorescence (ex/em=470 nm/525 nm) mode (FIG. 12). The green labelling of cell nucleic acids by Syto 9 allows discriminating intact bacteria (green rod shapes on FIG. 12, left panel) from lysed cells (homogenous green droplet staining on FIG. 12, right panel). This experiment showed that not only the presence of the lysis agent does not challenge droplets stability, but it also allows to efficiently lyse the cells and release their content into the droplets. Both approaches confirmed that cells can be isolated and lysed in droplets in conditions that do not compromise droplets stability. Moreover, the lysis conditions (i.e. CutSmat® buffer supplemented with B-PER™) are compatible with several molecular biology reactions such as reverse transcription, DNA digestion by restriction enzymes and DNA ligation. This example demonstrates the possibility of lysing cells into the droplets while preserving droplet integrity and being compatible with downstream molecular biology reactions.

### Example 2: Preparation of the Unique Identifier and Droplet Signatures

In this example, we show how Unique Identifiers (UIs) can be prepared both in bulk and in microfluidic droplets as well as how they can be used to generate a signature encoding droplet identity. A UI is made of a pair of random sequences (the UEI barcode (“unique entity identifier”) and the UEI Calibrator barcode) surrounded by constant sequence regions encompassing restriction sites generating compatible extremities later used to recombine both sequences together (FIG. 13). To form a UI, both UEI-Calibrator and UEI sequences should be first amplified in a duplex PCR prior to being recombined together through a specific restriction/ligation reaction. Then, the pool of UI contained into the same droplet constitute the signature of the droplet that can later be used to reassign a given encoded molecule to the droplet it originates from.

a. Duplex PCR Co-Amplification of the Two Barcode Sets

We first validated the possibility of performing a duplex PCR co-amplification of UEI-Calibrator and UEI sequences in tubes prior to transferring it in droplet microfluidic format.

Experimental Procedure

500 atto moles of one or both templates (i.e. UEI-DraIII (1) and AlwNI-Calibrator-DraIII (4)) diluted into 0.2 mg/mL yeast total RNA (Ambion) were introduced into a reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA) in a final volume of 50 μL. The mixture was then thermocycled with an initial denaturation step of 30 sec at 95° C. followed by 25 cycles of 5 sec at 95° C. and 30 sec at 65° C.

Results

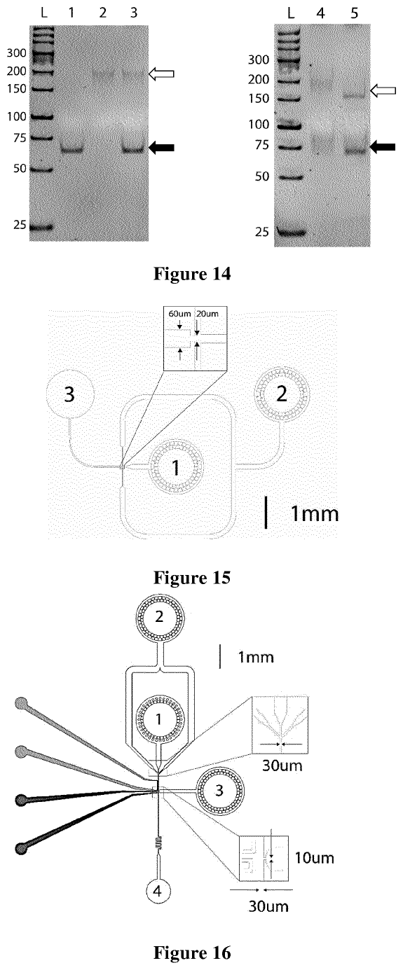

Upon amplification, PCR products with the expected size (62 base pairs for UEI-DraIII and 142 base pairs for AlwNI-Calibrator-DraIII) were readily observed on 8% polyacrylamide native gel. Whereas, a single band was observed when only one of the template was present (FIG. 14, lane 1 and 2), two distinguished bands were seen when both templates were mixed together (FIG. 14, lane 3), confirming that both templates can be efficiently co-amplified. In this section, only the Calibrators carried a randomized region, which explains the slightly smeary aspect of the band corresponding to the Calibrator. This smear was attributed to the formation of heteroduplexes between the strands of two different Calibrators sharing the same constant regions but having different randomized ones.

b. Amplification of the PCR in Water-in-Oil Droplet

We next verified that the co-amplification can efficiently occur in droplets.

Experimental Procedure

14 atto moles (a concentration allowing for having a theoretical average of 4 template DNA molecules per droplet) of each template (i.e. Template UEI-N15-DraIII (7) and Template AlwNI-N15-Calibrator-DraIII (8)) diluted into 0.2 mg/mL yeast total RNA (Ambion) were introduced in 200 μL of reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA. The mixture was then loaded into a length of PTFE tubing (I.D. 0.75 mm tubing; Thermo Scientific) and connected to the Fluigent infusion device at one side of the tubing while the other the other side was connected to a 40 μm deep droplet generator (FIG. 15, inlet 1). An oil phase made of Novec 7500 supplemented with 3% Krytox-Jeffamine 1000 diblock copolymer fluorosurfactant (Ryckelynck et al 2015) was also infused into the chip (FIG. 15, inlet 2) and used to produce 100 pL droplets at a rate of 1600 droplets per second by infusing oil and aqueous phases at 1500 nL/min and 1550 nL/min respectively. Droplets were collected for 11 min via a length of tubing (FIG. 15, outlet 3) into a 0.2 mL PCR tube closed by a plug of PDMS. The tube was then placed in a thermocycler and the emulsion was subjected to an initial denaturation step of 30 sec at 95° C. followed by 25 cycles of 5 sec at 95° C. and 30 sec at 65° C. Upon thermocycling, the emulsion was broken using 50 μL of 1H, 1H, 2H, 2H, perfluoro-1-octanol (Sigma-Aldrich) and 5 μL were loaded on an 8% native PAGE.

Results

Gel analysis confirmed that the duplex PCR amplification properly worked in water-in-oil droplets (FIG. 14, lane 5) and with the same apparent efficiency than in bulk (FIG. 14, lane 4). Moreover, the analysis revealed that compartmentalizing the reaction into droplets allowed obtaining better resolved bands, likely by limiting recombination, the formation of heteroduplexes and other PCR artefacts linked to the presence of the randomized regions on both barcodes (UEI-Calibrators and UEIs).

c. UI-Based Signature

Forming the UI requires recombining together the UEI-Calibrator and the UEI through a restriction/ligation coupled reaction. To do so, we choose two non-palindromic restriction sites producing compatible 3′ overhangs. AlwNI and DraIII are two enzymes digesting sequences (bolded in Table 1) fulfilling these criteria. Digestion at these sites generates two compatible sequences that can recombine together and form new sequences that can no longer be digested by the enzymes, pulling therefore the equilibrium toward the formation of recombined molecules.

Experimental Procedure

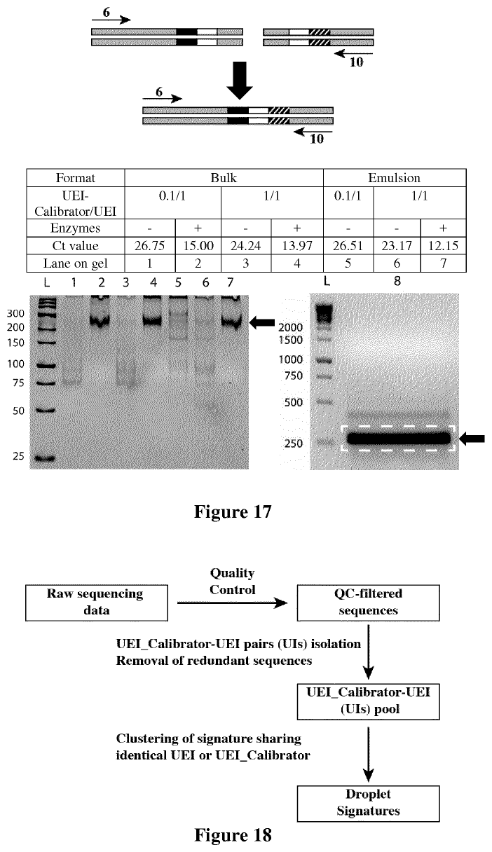

The duplex PCR established in section b was repeated using template molecules 7 and 8 and amplification primers 3, 5 and 6. However, primer 2 was exchanged for primer 9 that allowed introducing Illu-1 sequence, an adaptor sequence later used for next generation sequencing analysis. To evaluate to what extend the relative amount of both barcodes can affect recombination efficiency, so UI formation, we performed a set of experiments using two relative concentrations of UEI-Calibrators and UEI in the droplets. This ratio was changed by varying the concentration of the corresponding primers in the PCR mixture. However, the diversity of the barcodes was preserved by initiating each experiment with the same average number of template per PCR droplet. Therefore, in a first reaction set 14 atto moles of each template diluted into 0.2 mg/mL yeast total RNA (Ambion) were mixed with 0.2 μM of each primer (3, 5, 6 and 10) giving a final ratio UEI-Calibrator/UEI of 1/1. In a second set of reactions, 14 atto moles of each template diluted into 0.2 mg/mL yeast total RNA (Ambion) were mixed with 0.2 μM of primers 3 and 10 and 0.02 μM of primers 5 and 6, giving a final ratio UEI-Calibrator/UEI of 0.1/1. Each template/primers mixture was then introduced into 200 μL of reaction mixture containing 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA). The mixture was dispersed into 100 pL droplets as above and the emulsion was collected for 20 min in a 0.2 mL tube closed by a plug of PDMS and thermocycled as before. Upon thermocycling, the emulsion was reinjected into a droplet picoinjector (FIG. 16, inlet 1) at 500 nL/min and the droplets were spaced by a stream of Novec 7500 oil supplemented with 2% Krytox-Jeffamine 1000 diblock copolymer fluorosurfactant infused at 1600 nL/min through a second inlet (FIG. 16, inlet 2). A mixture of CutSmart™ buffer 1× (50 mM Potassium acetate, 20 mM Tris acetate pH7.9 at 25° C., 10 mM Magnesium acetate and 0.1 mg/mL BSA), supplemented with 1 mM rATP, 7 mM DTT, 3 U/μL DraIII HF (New England Biolabs), 3 U/μL AlwN1 (New England Biolabs), 30 U/μL T4 DNA ligase (New England Biolabs), 20 μM coumarin acetic (used as injection tracker) and 0.1% pluronic F68 was prepared. The mixture was loaded into a length of PTFE tubing (I.D. 0.75 mm tubing; Thermo Scientific) and connected to the Fluigent infusion device at one side of the tubing and to the third inlet of the chip (FIG. 16, inlet 3) at the other side. Then, 25 pL of the enzyme mixture were delivered to each 100 pL droplet by infusing the “enzyme” phase at 150 nL/min while energizing the electrodes facing the injection point with a squared AC field (400 V, 30 Hz) obtained by a function generator connected to an high voltage amplifier (TREK Model 623B). Droplets were collected (FIG. 16, outlet 4) in a 0.5 mL tube under mineral oil for 45 minutes prior to being incubated overnight at 37° C. to allow digestion and ligation to occur. In parallel, an identical reaction in which enzymes were omitted was performed as a control. Upon incubation, the emulsion was broken with 50 μL of 1H, 1H, 2H, 2H, perfluoro-1-octanol (Sigma-Aldrich) and the labelling efficiency was assessed by quantitative PCR with the kit SsoFast Evagreen Supermix (Bio-Rad) supplemented with primers 6 and 10. The mixture was thermocycled in a CFX qPCR machine (Bio-Rad) with an initial denaturation step of 30 sec at 95° C. followed by 40 cycles of 5 sec at 95° C. and 30 sec at 60° C. At the end of the process, the Ct value was determined for each condition (FIG. 17, Top and middle panels) and the quality of the amplified material was verified by loading an aliquot of each qPCR reaction onto a native polyacrylamide gel (FIG. 17, bottom left panel). After having verified the quality of the DNA, ˜1000 droplets from the emulsion containing the 1/1 UEI-Calibrator/UEI ratio were transferred in a new tube where they were broken with 50 μL of 1H, 1H, 2H, 2H, perfluoro-1-octanol (Sigma-Aldrich) and the released DNA was recovered in 1× CutSmart™ buffer. The DNA was then indexed with primers N503 and N702 using Nextera index kit (ref FC-121-1011, Illumina) following supplier specifications. To limit the risk of unwanted mutations during the indexing step, the reaction was performed using Phusion DNA polymerase (ThermoScientific). The band containing indexed DNA was then purified on a 1% agarose gel electrophoresis and the DNA recovered with the kit Wizard SV Gel and PCR clean up system (Promega) (FIG. 17, right panel). Last, the resulting library was loaded onto an Illumina V2-300 cycles flow-cell and the DNA was sequenced on a MiSeq device (Illumina).

Finally, the sequences were analyzed using a Python-based bioinformatics algorithm (FIG. 18). The algorithm works in 3 main steps. First, raw sequences obtained from the sequencer are quality-filtered and those with a Q score below 30 are removed from the pool. Moreover, sequences presenting mutations in the non-randomized region or showing an inappropriate length are also removed from the pool. Second, UEI-Calibrator and UEI sequences are extracted and pairs coming from the same droplet are clustered together. Briefly, all the UEIs associated with a given UEI-Calibrator are clustered together. Then, all the UEI-calibrator sharing the UEIs contained into the same cluster are also clustered together. At the end of the process, clusters of UIs (pairs of UEI-Calibrators and UEIs) are obtained and form a signature of the droplet. Finally, in a third step, the signatures can be used to reassign each molecule from a pool to the droplet it originates from.

Results

The UI signature readily forms only in the presence of restriction/ligation enzymes. Indeed, the qPCR analysis revealed that whatever the UEI-Calibrator/UEI ratio used, more than 10 additional amplification cycles (delta Ct>10) are required the reach the threshold in the absence of recombination enzymes (FIG. 17, Top panel, compare columns − and + enzymes), indicating that there is at least a thousand times less recombined material in the absence of enzymes. Moreover, electrophoresis gel analysis (FIG. 17, left panel) showed that specific band of the expected size is obtained only in the presence of the enzymes (lanes 2, 4 and 7). Therefore, UI formation is highly controlled as it occurs only in the presence of specific enzymes and does not form spontaneously form during the PCR reaction, which limits the risk of forming UI in non-specific way. Not only this recombination was found to work in tubes (FIG. 17, left panel, lanes 1 to 4) but it also works in droplets (FIG. 17, left panel, lanes 5 to 7) starting from PCR amplification products also prepared in droplets. As in tubes, the presence of UI in droplets requires the presence of the specific restriction/ligation enzymes (compare lanes 6 and 7).

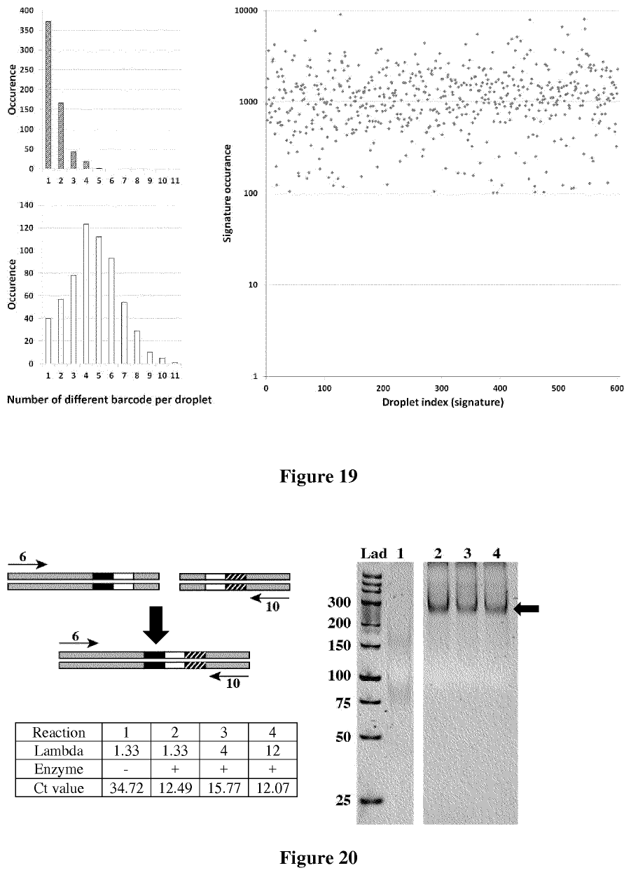

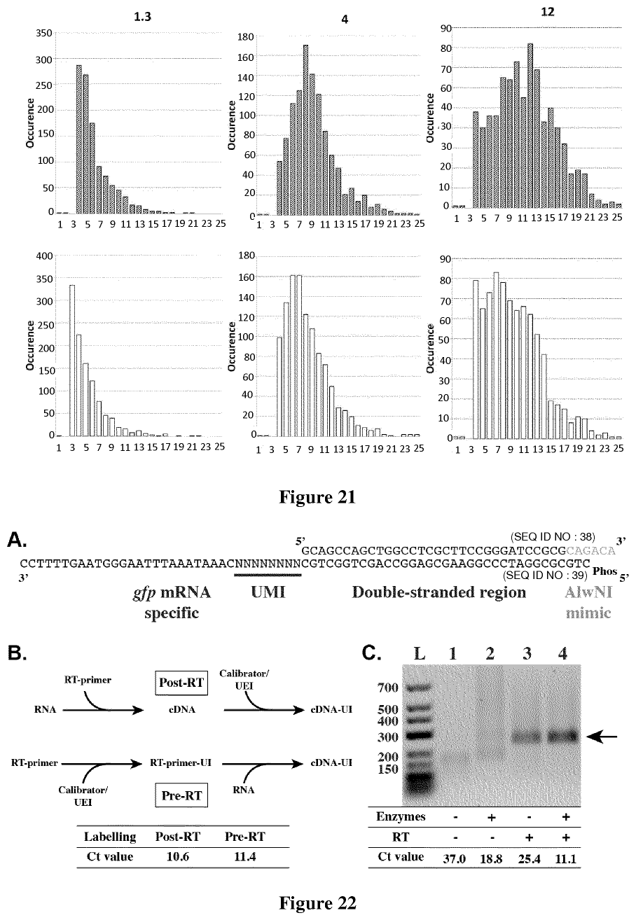

Further analyzing the droplet-contained UI by Next Generation Sequencing together with the bioinformatics algorithm shown on FIG. 18, allowed us to reassign each UEI and each UEI-Calibrator to the droplet it originates from (FIG. 19). Indeed, the use of a barcode made of 15 randomized (theoretical diversity of 10⁹ different variants) region makes unlikely to find the same UEI or the same UEI-Calibrator in more than one droplet in the emulsion (made at most of a few millions of droplets), making this reassignment faithful. The number of different UEI and UEI-Calibrator per droplet followed a Poisson distribution (FIG. 19, left; see also FIG. 21) as expected for objects randomly distributed into compartments such as droplets. This confirms that the proper confinement of the information was preserved all along the process and that the UI were generated in the droplets and not after droplet breaking. Note that in this example, the average number of different UEI-Calibrator per droplet was only 1 while 4 were expected. However, complementary experiments presented hereafter (section d) allowed readjusting this parameter and further confirming the Poisson-driven behavior of the molecules.

Clustering the different UIs contained in each droplet allows obtaining a signature unique to each droplet. Interestingly, the signatures were evenly represented within the pool of sequences (FIG. 19, right), indicating that no significant droplet-to-droplet bias occurs during UI preparation.

d. Adjusting UEI Diversity

To further confirm that both UEI-Calibrators and UEIs distributes into the droplets following a Poisson distribution, as well as to properly adjust the content of each type of barcodes, the experiment was performed at three different average number (lambda values) of barcode per droplet.

Experimental Procedure

As before, a duplex PCR was performed but using different starting amount of template molecules 7 and 8. To test the lambda 1.3 condition (i.e. an average of 1.3 UEI-Calibrator and 1.3 UEI per droplet), 5 atto moles of each template (i.e. Template UEI-N15-DraIII (7) and Template AlwNI-N15-Calibrator-DraIII (8)) diluted into 0.2 mg/mL yeast total RNA (Ambion) were introduced into 200 μL of a reaction mixture containing 0.2 μM of each forward (molecules 2 and 6) and reverse (molecules 3 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA. To test the lambda 4 (i.e. an average of 4 different UEI-Calibrators and 4 different UEIs per droplet) and lambda 12 (i.e. an average of 12 different UEI-Calibrators and 12 different UEIs per droplet) conditions, the starting amount of each template introduced in the reaction mixture was respectively raised to 14 atto moles and 56 atto moles. 100 pL droplets were generated at a frequency of 300 droplets per second using the same chip as before (FIG. 15) and the emulsion was collected and thermocycled as in section c. Finally, as above, an enzyme mixture was picoinjected into each droplet and the emulsion was incubated to allow the recombination to occur. Moreover, a control reaction in which restriction/ligation enzymes were omitted was performed in parallel with the lambda 1.3 condition. Upon incubation, the proper recombination was again verified by qPCR (using primers 6 and 10) and gel electrophoresis (FIG. 20). Moreover, for each condition, ˜1000 droplets were transferred into a new tube where they were broken by adding 20 μL of 1H, 1H, 2H, 2H, perfluoro-1-octanol (Sigma-Aldrich) and the released DNA was recovered in 200 μL of PCR mixture containing Q5 DNA polymerase (New England Biolabs) and its buffer at the recommended concentration, 0.2 mM of each dNTP and a Nextera primer pair (N703 and N502 to index the lambda 1.3 condition; N705 and N504 to index the lambda 4 condition; N706 and N517 to index the lambda 12 condition; Illumina) at the recommended concentration. Indexing was performed by thermocycling the mixture using the program recommended by the manufacturer and the amplification product was loaded on a 1% agarose gel). Upon electrophoresis, the band containing the indexed DNA was sliced from the gel and the DNA was recovered with the kit Wizard SV Gel and PCR clean up system (Promega). Finally, the resulting library was loaded into an Illumina V2-300 cycles flow-cell and a high-throughput sequencing was performed on a MiSeq device (Illumina). Then, sequencing data were analyzed using our bioinformatics algorithm (FIG. 18) and the distribution profiles of both UEI-Calibrators and UEIs were established for each condition (FIG. 21).

Results

Preparing UIs in droplets while varying the initial number of UEI-Calibrator and UEI template per droplet (lambda value) did not significantly challenged the process per se. Indeed, whatever the starting lambda value (from 1.3 to 12), very close Ct values were obtained when quantifying the amount of DNA amplified and recombined into the droplets at the of the process (reactions 2 to 3 on FIG. 20). With respect to the enzyme-free control (reaction 1 on FIG. 20), up to 20 less amplification cycles were required to reach the threshold when enzymes were added, indicating that there was up to a million times more UI formed in the presence of enzymes. Analysis on gel, confirmed that the proper reaction product (indicated by an arrow on FIG. 20) was observed only in the presence of the enzyme (lanes 2 to 4), whereas only a small parasitic amplification product was obtained in the absence of enzymes (lane 1). The smeary aspect of the bands was likely due to heteroduplex formation occurring at the end of the PCR process.

Analyzing the sequence content of an aliquot of 1000 droplets from each condition with our bioinformatics algorithm revealed that, as expected, changing the starting amount of UEI-Calibrator and UEI template per droplet directly impacts the distribution of both barcodes in the droplets (FIG. 21) and that the higher the starting number of different UEI-Calibrator and UEI per droplet the higher the number of different UI forming droplet signature. Therefore, adjusting the initial concentration of both templates allows adjusting the diversity of UI per droplet (so the complexity of the signature) in a predictable way.

### Example 3: Preparing cDNAs Using RT Primers Pre-Labelled with UIs

Since UIs are obtained through a digestion/ligation process, they can also be appended to another DNA molecule provided it possesses a site compatible with one of the restriction products. To do so, we designed a composite reverse transcription (RT) primer made of i) double-stranded 5′ part terminated by a 3′ overhang compatible with the restriction site present at the extremity of the UEI/UEI-calibrator sequence; followed by ii) a single-stranded region containing 8 random nucleotides and working as Unique Molecular Identifier (UMI barcode) and iii) terminated by 3′ single-stranded region annealing specifically to the target RNA (FIG. 22A).

In this example, we demonstrate that a target nucleic acid molecule (GFP-coding mRNA) contained in droplets can be efficiently converted into a cDNA while using an RT primer comprising UMI, UEI and UEI-calibrator barcodes.

We tested this approach using the messenger RNA coding for the Green Fluorescent Protein (GFP). This mRNA was isolated from a pellet of bacteria overexpressing the gene coding for the GFP by a Trizol extraction following manufacturer recommendation. We then designed an RT primer (molecule 23) containing the sequences required for the addition of a UEI/UEI-calibrator sequence and containing a UMI barcode. We next tested if grafting this sequence to the RT primer still allowed an efficient reverse transcription of the target RNA to occur.

First a duplex PCR during which the UEI-Calibrator sequences and the UEI sequences were co-amplified (as described in Example 2) using 500 atto moles of each template (11 and 15) as well as 0.2 μM of each primer (2, 5, 10 and 12). After thermocycling, 37.5 μL of this mixture was supplemented with 0.46 pmol of the RT primer (molecule 23), 0.46 pmol of the primer complementary to its 5′ end (molecule 14), 1.5 U of DraIII (New England Biolabs), 1.5 U of AlwNI (New England Biolabs), 150 U of T4 DNA ligase (New England Biolabs), 1 mM rATP, 0.02% Pluronic F68 and DTT 10 mM contained in 1× CutSmart® buffer. The mixture was then subjected to 5 cycles of temperature: 15 min at 37° C. and 45 min at 16° C.

Upon incubation, the mixture was further supplemented with 2 μL of Trizol-extracted total RNA (containing gfp mRNA), 1 mM each dNTP and 10 U of AMV reverse transcriptase. The mixture was then incubated for 1 hour at 42° C.

In a first set of controls, the experiment was repeated while omitting either the restriction/ligation enzymes, or the reverse transcriptase or both. In a second control experiment, the same amount of Trizol-extracted total RNA gfp mRNA was reverse transcribed prior to labeling the cDNA with UEIs following the procedure described in Example 2.

Finally, reaction efficiencies were determined by qPCR using the SsoFast Evagreen Supermix kit (Bio-Rad) supplemented with primers 24 and 10, while taking care of introducing the same theoretical amount of RNA (expected to be converted into cDNA). Moreover, the amplification products were analyzed on a 1% agarose gel (FIG. 22C).

Results

In this example, we found that using prelabeled RT primers does not affect their functionality since the same amount of cDNA was generated in the presence and in the absence of the label (nearly identical Ct values with pre and post-RT labeling on FIG. 22B). Moreover, the presence of both the reverse transcriptase and the recombination enzymes was required to obtain cDNA displaying the expected size (pointed by the arrow on FIG. 22C). The presence of a band of the expected size on lane 3 was attributed to a slight cross contamination during the experiment. Yet, the difference of 14 cycles in the Ct values of both samples (lane 3 and 4) indicated that the unwanted product was present at a concentration ˜16,000 (2¹⁴) lower in the control (lane 3) than in the assay (lane 4). Furthermore, even though the Ct value obtained in the presence of the RT only (reaction 2) was closer to that of the assay, the gel analysis revealed that this signal was mainly due to non-specific products (see the smear on FIG. 22C, lane 2). Overall, this example demonstrates that pre-labeling RT primer using the method presented here does preserve their functionality.

### Example 4: Establishing the Capacity of Converting Low Abundant RNAs into Encodable cDNA in Emulsion Droplets

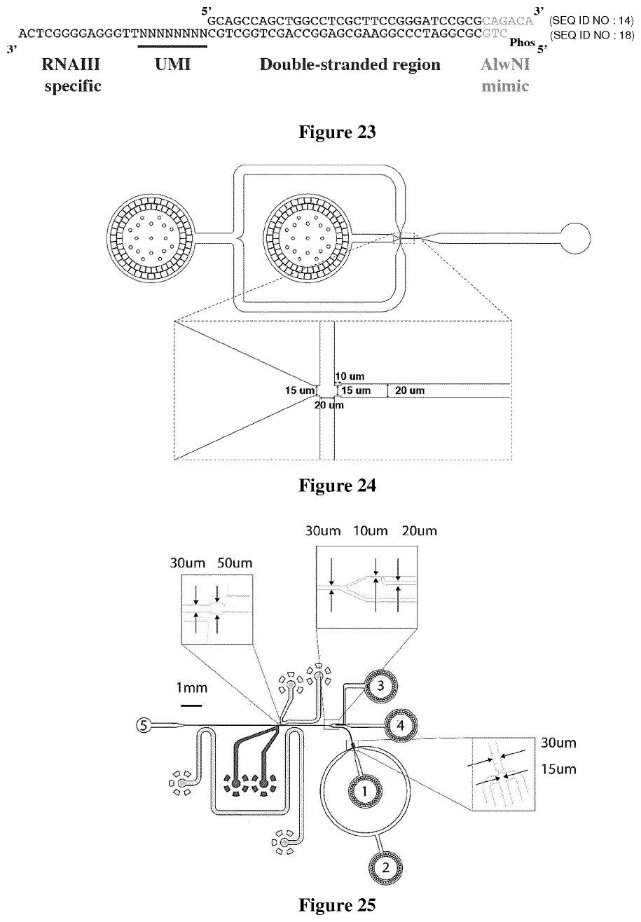

Thought we showed in example 3 that using composite RT primers does not compromise its functionality, in this example we extended the concept to a more structured RNA, therefore more difficult to reverse transcribe that less structured mRNA such as gfp mRNA used in example 3. We choose RNA III from Staphylococcus aureus (Benito et al, 2000), a non-coding RNA adopting a compact multidomain folding. As a first step, we prepared an RNA III specific composite RT primer (FIG. 23) and then determined if as few as 10 copies of RNA III per droplet could still be efficiently reverse transcribed and recovered from emulsion.

RNA III was prepared by in vitro transcription using T7 RNA polymerase using the same procedure described in (Ryckelynck et al, 2015). Upon transcription, RNA was purified on a size exclusion column Nap5 (GE-Healthcare) and quantified using a Nanodrop device. To prevent unwanted reverse transcription of the RNA prior to its encapsulation in droplets, RNA and RT reagents were first emulsified separately and mixed together through droplet fusion (Mazutis et al, 2009). Moreover, to evaluate the sensitivity of the reverse transcription process, we prepared emulsions containing 10, 100 or 1000 RNA molecules per droplet. Then the reverse transcription was added to each droplet and the RT allowed to proceed prior to analyzing the produced cDNA.

Experimental Procedure

0.6, 6 or 60 femto moles of RNA-III (allowing for having respectively, 10, 100 or 1000 RNA molecule per 2 pL droplet) were introduced into a 100 μL of a solution containing CutSmart® buffer, 1.5 mg/mL Dextran Texas Red (Invitrogen), 0.25% Pluronic F68. The mixture was then loaded into a length of PTFE tubing (I.D. 0.75 mm tubing; Thermo Scientific) and connected to the Fluigent infusion device at one side of the tubing while the other the other side was connected to a 10 μm deep droplet generator (FIG. 24, inlet 1). An oil phase made of Novec 7500 supplemented with 3% Krytox-Jeffamine 1000 diblock copolymer fluorosurfactant was also infused into the chip (FIG. 24, inlet 2) and used to produce 2 pL droplets at a rate of 9000 droplets per second by infusing oil and aqueous phases at 1450 nL/min and 700 nL/min respectively. Droplets were collected (FIG. 24, outlet 3) via a length of tubing into a 0.2 mL PCR tube closed by a plug of PDMS. 200 μL of reverse transcription mixture were prepared by supplementing a CutSmart® buffer prepared at the recommended concentration with 1.25 pmols of RT-UMI-RNAIII primer (molecule 18), 2.5 pmol of Anti-SBACA primer (molecule 14), 0.25 mM of each dNTP, 10 mM DTT, 0.1 μM FAM (droplet tracker) and 125 U of Reverse Transcriptase Maxima RNase H minus (Thermo Scientific). The mixture was loaded into a length of PTFE tubing (I.D. 0.75 mm tubing; Thermo Scientific) and connected to the Fluigent infusion device at one side of the tubing while the other the other side was connected to a 15 μm deep droplet fusion device (FIG. 25, inlet 1). An oil phase made of Novec 7500 supplemented with 2% Krytox-Jeffamine 1000 diblock copolymer fluorosurfactant was also infused into the chip (FIG. 25, inlet 2) and used to produce 18 pL droplets at a frequency of 1200 droplets per second by infusing oil and aqueous phases at 1280 nL/min and 800 nL/min respectively. Moreover, the 2 pL droplets containing the RNA were also reinjected into the same chip (FIG. 25, inlet 3) at a frequency of ˜1200 droplets per second by infusing them at 140 nL/min and spacing them with a stream of surfactant-free Novec 7500 fluorinated oil (3M) infused into the chip (FIG. 25, inlet 4) at 1400 nL/min. Pairs of droplet were formed and fused by a squared AC field (450 V, 30 Hz) that was applied to built-in electrodes and obtained from a function generator connected to a high voltage amplifier (TREK Model 623B). Fused droplets were then collected (FIG. 25, outlet 5) under mineral oil. In parallel, the same reaction was performed in bulk by mixing 1 volume of RNA dilution with 9 volumes of reverse transcription mixture and by incubating both the bulk mixture and the emulsions for 1 hour at 55° C. Furthermore, a second control reaction in which the reverse transcriptase was omitted was also performed. Upon incubation, emulsions were broken using of 1H, 1H, 2H, 2H, perfluoro-1-octanol (Sigma-Aldrich) and the aqueous phases were recovered. cDNA obtained from the different conditions (bulk or emulsion; 10, 100 or 1000 RNAs per droplet) was then analyzed by qPCR using the SsoFast Evagreen Supermix kit (Bio-Rad) supplemented with the primers 20 and 21. Moreover, the amplification products were analyzed on an 8% native polyacrylamide gel.

Results

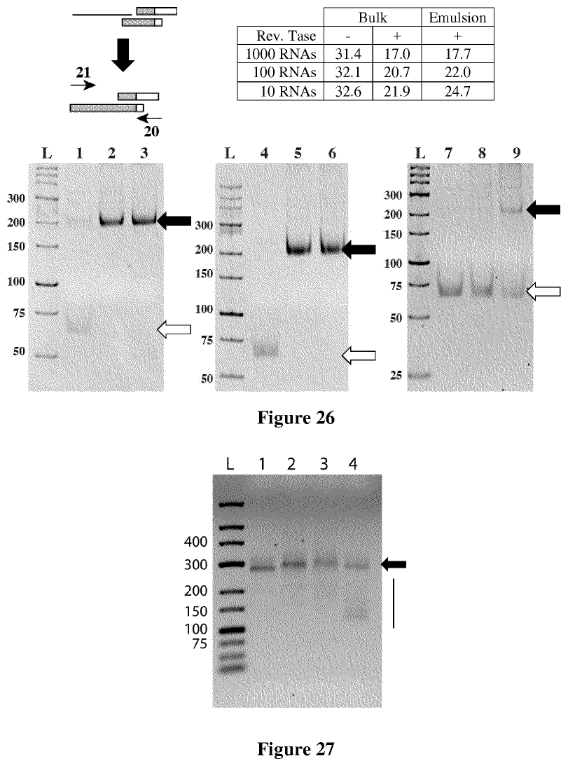

In this example, emulsions of 2 pL droplets containing each 10, 100 or 1000 molecules of RNA-III were produced and fused to 18 pL droplets containing an RT mixture. As a control, the same experiment was performed in a bulk format. Upon incubation, qPCR analysis revealed that RT occurred with the same efficiency both in bulk and emulsified format (FIG. 26). Indeed, in both formats Ct values obtained with bulk and emulsified reactions were very close and significantly higher than that of a control reaction where the reverse transcriptase was omitted. Moreover, the analysis on gel confirmed that the qPCR product obtained in both conditions had the expected size, whereas only a low size PCR side product was obtained with the negative control. Interestingly, the condition where the RNA was the most diluted (10 molecules per 2 pL) gave a detectable signal corresponding to an amplification product of the expected size only in emulsion demonstrating the higher sensitivity offered by the droplet format.

### Example 5: Exploring Alternative Format of UEI-Calibrator and UEI Sequences

We noticed that, when working at low concentration of target molecules, some non-specific recombination events occur between some primers and the randomized regions of the UEI-Calibrators and the UEIs. We therefore explored alternative sequences to the simple stretch of 15 contiguous randomized nucleotides. In this example, we reverse transcribed a purified gfp mRNA prior to labelling the obtained cDNA with UEI and UEI-calibrator sequences produced from templates: i) deprived of randomized regions; ii) or bearing a stretch of 15 contiguous randomized nucleotides (N15); iii) or bearing a stretch of semi-randomized nucleotides where 5 randomized dinucleotides are spaced by constant dinucleotides (N2x5); iv) or bearing a stretch of semi-randomized nucleotides where 4 randomized trinucleotides are spaced by constant trinucleotides (N4443). We then evaluated the capacity of each sequence design to generate a clean labelled cDNA obtained from the gfp mRNA.

Experimental Procedure

gfp mRNA was prepared by in vitro transcription using T7 RNA polymerase using the same procedure described in (Ryckelynck et al, 2015). Upon transcription, RNA was purified on a size exclusion column Nap5 (GE-Healthcare) and quantified using a Nanodrop device. 16.67 femto moles of purified gfp mRNA were reverse transcribed in 20 μL CutSmart® buffer prepared at the recommended concentration and supplemented with 1.25 pmol of RT primer (molecule 23), 1.25 pmol of the complementary oligonucleotide (molecule 24), 1 mM dNTP, 10 mM DTT, 10 U of AMV reverse transcriptase, 0.1 μM FAM. The mixture was then incubated 1 hour at 42° C. 500 atto moles of pairs of template UEI-Calibrators and UEIs (i.e. 1/27, 25/28, 26/29 and 7/30) were co-amplified in 200 μL of amplification mixture supplemented with 0.2 μM of each primer (2, 5, 10 and 12) as described in Example 2. Upon thermocycling, 10 μL duplex PCR were mixed with 4 μL of reverse transcription reaction and 3 μL of Enzyme mixture (1× CutSmart® buffer with 6 mM rATP, 60 mM DTT, 8 U/μL DraIII HF, 8 U/μL AlwN1, 80 U/μL T4 DNA ligase, 55 μM coumarin acetic and 0.3% pluronic F68). The mixture was then subjected to 5 cycles of temperature: 15 min at 37° C. and 45 min at 16° C. At the end of the incubation, 1 μL of each reaction was amplified by PCR using the kit Sso-Fast Evagreen (Bio-Rad) supplemented in primers 3 and 6 and the amplification products were analyzed on a 1% agarose gel (FIG. 27).

Results & Conclusions

The target RNA (gfp mRNA) was properly reversed transcribed and the 4 types of barcodes (UEI-Calibrator/UEI) were appended to the resulting cDNA. Whereas barcodes deprived of randomized regions or affording N2x5 semi-randomized regions gave homogeneous PCR products (lanes 1 and 2 on FIG. 27), more smeary bands were observed with the N4443 and N15 regions. Moreover, secondary amplification products of smaller size tend to significantly accumulate with N4443 and N15 (lanes 3 and 4 on FIG. 27). Therefore, semi-randomized barcodes such as the N2x5 represent an attractive alternative to the more conventional N15 barcode design.

### Example 6: Labelling Proteins with Identification Sequences

To extend the use of the technology to molecules other than nucleic acids, we validated in this example the capacity of our method to produce probes comprising a protein as capture moiety. To this end, we cloned a fusion gene made of protein A and SNAP tag (New England Biolabs) coding regions. The resulting gene was overexpressed in E. coli and the resulting protein (NaBAb) was purified. In this example, we verified that a unique fragment of DNA comprising an identification sequence could be covalently attached to the protein and that it did not interfere with the capacity of the protein to interact with antibodies via its protein A moiety.

a. Labelling Antibodies with a Single DNA Per Antibody

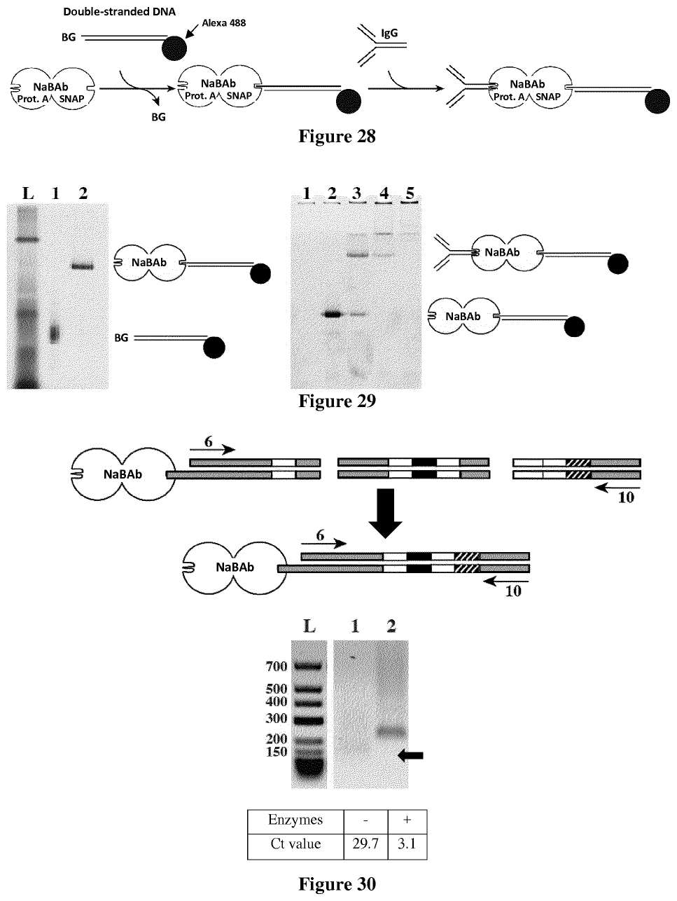

We prepared a construct in which the sequence coding for the protein A was placed in fusion with a His-tagged version of the SNAP-tag (New England Biolabs) and placed the construct on an expression plasmid. The corresponding protein was then overproduced in E. coli and purified by affinity chromatography. The SNAP domain is a catalytic module reacting with benzyl-guanine-displaying substrate molecules. Upon reaction, a single molecule of substrate is covalently and irreversibly attached to the SNAP module (FIG. 28). Moreover, this protein modified with a single DNA molecule should also be able to strongly associate with antibodies via its protein A domain. In this work, we show that our fusion protein is indeed able to be labelled by a single DNA molecule and that the resulting complex is still able to interact with antibodies.

Experimental Procedure

A template DNA (molecule 31) possessing a unique AlwNI as well as a UMI of 8 randomized positions was PCR amplified using a primer modified with a benzyl-guanine (BG) group (molecule 32) allowing for grafting the DNA on the SNAP module of NaBAb and a primer modified with an Alexa-488 fluorescent group (molecule 33) to fluorescently label the DNA. 10 pmols of template 31 were mixed with 1 nmol of each primer (32 and 33) in 1 mL of PCR solution containing 0.2 mM of each dNTP, 20 U of Q5 DNA polymerase (New England Biolabs) and the corresponding buffer at the recommended concentration. The mixture was then placed in thermocycler and subjected to an initial denaturation step of 30 sec at 98° C. followed by 25 cycles of 10 sec at 98° C., 10 sec at 55° C. and 30 sec at 72° C. Finally, the program was concluded by a final extension step of 2 min at 72° C. Amplification products were the purified using the kit Wizard SV Gel and PCR clean up system (Promega) and quantified with a Nanodrop device. 200 pmols of BG/Alexa488 dually labelled DNA were then mixed with 180 pmol of purified NaBAb in 1 mL of CutSmart® buffer (New England Biolabs) diluted at the recommended concentration (1×) and supplemented with 1 mM DTT. The same experiment in which NaBAb was omitted was performed in parallel and used as control. The mixture was incubated for an hour at 37° C. and an aliquot was analyzed on polyacrylamide gel electrophoresis and Alexa488 fluorescence revealed (FIG. 29, left panel).

In a second experiment, 27 pmols of purified NaBAb were mixed with 20 pmols of BG/Alexa488 dually labelled DNA in 40 μL of CutSmart® buffer (New England Biolabs) diluted at the recommended concentration (1×) and supplemented with 1 mM DTT. Moreover, the mixture was also supplemented with 225, 112, 62.5 μg/mL of total Human IgG (Sigma Aldrich). Reactions in which IgG and/or the dually labelled DNA were omitted were used as control. The mixture was incubated for an hour at 37° C. and an aliquot was analyzed on polyacrylamide gel electrophoresis and Alexa488 fluorescence revealed (FIG. 29, right panel).

Results & Conclusions

Incubating BG/Alexa488 dually labelled DNA with a slight excess of NaBAb allowed grafting all the DNA onto the SNAP module of NaBAb as attested by the complete up-shift of the DNA band on the gel (FIG. 29, left panel). Forming the NaBAb-DNA covalent complex in the presence of IgG showed that NaBAb-DNA/IgG ternary complex can readily form (FIG. 29, right panel, lanes 3 to 5). Moreover, considering the average molecular weight of IgG to be ˜150 kDa one can see that the 27 pmols of NaBAb-DNA complex are completely shifted in the presence of an equimolar amount of IgG (125 mg/mL or 30 pmols IgG, lane 4) whereas only half of the complex is shifted using twice less IgG. These data indicate that not only NaBAb can be covalently linked to a single DNA molecule but mixing it stoichiometrically with an IgG allows forming a NaBAb-DNA/IgG ternary complex in which one antibody molecule is predominantly labelled with a single DNA molecule.

b. Assembling NaBAb-DNA Complex with UEI/UEI-Calibrator Sequences

We investigated the possibility of assembling the NaBAb-DNA complex with UEI/UEI-calibrator sequences.

Experimental Procedure

500 atto moles of template UEI-Calibrators and UEIs (molecules 11 and 15) were co-amplified in 200 μL of PCR mixture supplemented with 0.2 μM of each primer (2, 5, 10 and 12) as described in Example 2. Upon thermocycling, 10 μL of duplex PCR were added to 10 μL of CutSmart® buffer (New England Biolabs) diluted at the recommended concentration (1×) supplemented with 2 pmol of NaBAb-DNA complex (prepared as described in section a of this Example), 2 mM rATP, 20 mM DTT, 2.5 DraIII HF (New England Biolabs), 2.5 U/μL AlwN1 (New England Biolabs), 25 U/μL T4 DNA ligase (New England Biolabs), 10 μM coumarin acetic and 0.1% pluronic F68. The mixture was then subjected to 5 cycles of temperature: 15 min at 37° C. and 45 min at 16° C. In parallel, we performed a control reaction in which restriction/ligation enzymes were omitted. At the end of the incubation, 1 μL of each reaction was analyzed by quantitative PCR using the kit Sso-Fast Evagreen (Bio-Rad) supplemented in primers 6 and 10, and the amplification products were analyzed on a 1% agarose gel (FIG. 30).

Results & Conclusions

In this section, we showed that a DNA covalently associated to the NaBAb protein can serve as UEI/UEI-calibrator acceptor using or restriction/ligation strategy. Indeed, in absence of the enzymes more than 26 additional amplification cycles (delta Ct>26) were required the reach the threshold (FIG. 30, compare columns − and + enzymes), indicating that there is at least a 33 million-times less complexes formed in the absence of enzyme. Moreover, gel electrophoresis analysis (FIG. 30) showed that specific band of the expected size is obtained only in the presence of the enzymes (lane 2).

In conclusion, in this example, we demonstrated that the NaBAb fusion protein can be used to specifically label IgG with a unique DNA molecule comprising UMI, UEI and UEI-calibrator barcodes. By extension, it should be possible to apply the same UIs based strategy labelling to DNA molecules displayed at the surface of any protein

### Example 7: Preparation of Barcoded cDNA Libraries for Sequencing Analysis

In this last example, we demonstrate the functionality of the whole invention by reverse transcribing RNAs in droplets by using pre-labelled RT primers to produced labelled cDNAs later indexed and analysed by high throughput sequencing. As starting material, we used RNA III prepared like in example 4 and the duplex 14/18 introduced earlier and targeting RNA III (FIG. 23). In addition, in this example, the Illu2 adaptor (sequence of molecule 6) was directly included in the UEI template (Template UEI-N15-DraIII-Illu2, molecule 36).

Experimental Procedure

We first prepared labelled 14/18 duplex. To do so, we first prepared droplets containing a PCR mixture supplemented with an average of 4 different UEI templates and 4 different UEI-calibrator templates per droplet (see example 2). Yet, in the present example, duplex PCR amplification of UEI and UEI-Calibrators was performed in 4 pL droplets. To do so, ˜330 atto moles (a quantity allowing for having a theoretical average of 4 template DNA molecules per 4 pL droplet) of each template (i.e. Template UEI-N15-DraIII-Illu2 (36) and Template AlwNI-N15-Calibrator-DraIII (8)) diluted into 0.2 mg/mL yeast total RNA (Ambion) were introduced in 200 μL of reaction mixture containing 0.2 μM of each forward (molecules 2 and 12) and reverse (molecules 6 and 5) primers, 0.2 mM of each dNTP, 0.1% Pluronic F68, 0.67 mg/mL dextran Texas Red (Invitrogen), 6 nM of a Taq polymerase produced in the laboratory and 1× CutSmart® buffer (50 mM Potassium acetate, 20 mM Tris acetate pH7.9 at 25° C., 10 mM Magnesium acetate and 0.1 mg/mL BSA). The mixture was then loaded into a length of PTFE, tubing (I.D. 0.75 mm tubing; Thermo Scientific) and connected to the Fluigent infusion device at one side of the tubing while the other the other side was connected to a 10 μm deep droplet generator (FIG. 24, central inlet). An oil phase made of Novec 7500 supplemented with 3% Krytox-Jeffamine 1000 diblock copolymer fluorosurfactant (Ryckelynck et al 2015) was also infused into the chip (FIG. 24, left inlet) and used to produce 4 pL droplets at a rate of 3800 droplets per second by infusing oil and aqueous phases at 1800 nL/min and 600 nL/min respectively. Droplets were collected for 30 min via a length of tubing (FIG. 24, right outlet) into a 0.2 mL PCR tube closed by a plug of PDMS. The tube was then placed in a thermocycler and the emulsion was subjected to an initial denaturation step of 30 sec at 95° C. followed by 40 cycles of 5 sec at 95° C. and 30 sec at 60° C. Upon thermocycling, PCR droplets were reinjected into a droplet fuser (FIG. 25, inlet 3) at a frequency of ˜1200 droplets per second by infusing them at 90 nL/min and spacing them with a stream of surfactant-free Novec 7500 fluorinated oil (3M) infused into the chip (FIG. 25, inlet 4) at 900 nL/min. In parallel, 100 μL of reaction mixture were prepared by supplementing a CutSmart® buffer prepared at the recommended concentration with 100 pmols (˜1 million copies per 16 pL droplet) of RT-UMI-RNAIII (molecule 18), 100 pmol (˜1 million copies per 16 pL droplet) of antiSBACA-AlwN1 (molecule 14), 30 U of DraIII (New England Biolabs), 30 U of AlwNI (New England Biolabs), 160 U of T4 DNA ligase (New England Biolabs), 1 mM rATP, 0.02% Pluronic F68 and 10 mM DTT. The mixture was loaded into a length of PTFE tubing (I.D. 0.75 mm tubing; Thermo Scientific) and connected to the Fluigent infusion device at one side of the tubing while the other the other side was connected to a 15 μm deep droplet fusion device (FIG. 25, inlet 1). An oil phase made of Novec 7500 supplemented with 2% Krytox-Jeffamine 1000 diblock copolymer fluorosurfactant was also infused into the chip (FIG. 25, inlet 2) and used to produce 16 pL droplets at a rate of 1200 droplets per second by infusing oil and aqueous phases at 750 nL/min and 700 nL/min respectively. Pairs of droplets were formed (one-to-one pairing efficiency ≥80%) and fused by a squared AC field (600 V, 30 Hz) that was applied to built-in electrodes and obtained from a function generator connected to a high voltage amplifier (TREK Model 623B). Fused droplets were then collected (FIG. 25, outlet 5) for 30 minutes via a length of tubing into a 0.2 mL PCR tube closed by a plug of PDMS. The mixture was then subjected to 18 cycles of temperature: 15 min at 37° C. and 45 min at 16° C.

In parallel, a solution RNA III diluted in 1× CutSmart buffer (New England Biolabs) supplemented with 0.05% Pluronic F68 and 1.5 mg/mL Dextran-Texas Red was dispersed in 100 pL droplets using the 40 μm deep droplet generator (FIG. 15) already used in example 2. RNA dilution was adjusted to have on average 1 million copies per 100 pL and the solution infused into the chip (FIG. 15, inlet 1). An oil phase made of Novec 7500 supplemented with 3% Krytox-Jeffamine 1000 diblock copolymer fluorosurfactant (Ryckelynck et al 2015) was also infused into the chip (FIG. 15, inlet 2) and used to produce 100 pL droplets at a rate of 300 droplets per second by infusing oil and aqueous phases at 1150 nL/min and 900 nL/min respectively. Droplets were collected via a length of tubing (FIG. 15, outlet 3) into a 0.2 mL PCR tube closed by a plug of PDMS.

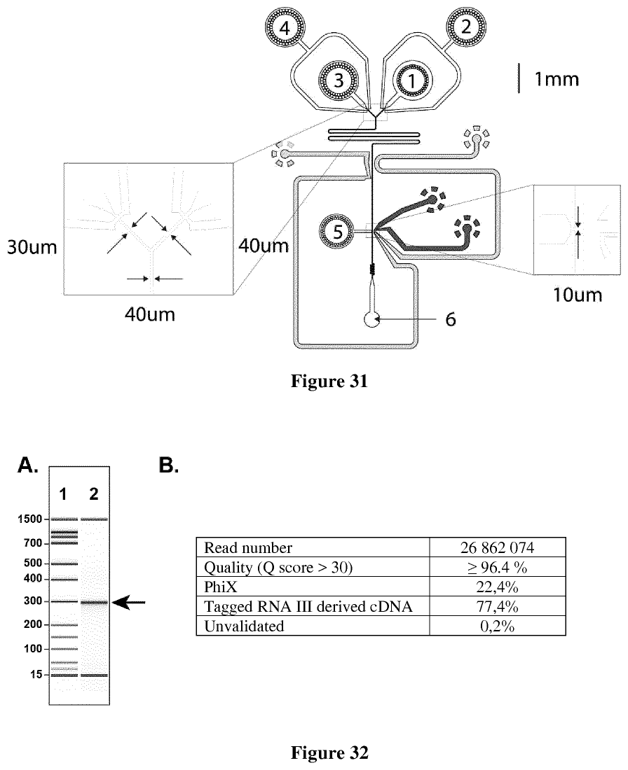

Upon incubation, both 20 pL (4 pL PCR droplets fused with 16 pL enzyme-containing droplets) and 100 pL droplets were reinjected into a new microfluidic device (FIG. 31). 20 pL droplets containing labelled RT primers were reinjected (FIG. 31, inlet 3) at a frequency of ˜100 droplets per second by infusing the emulsion at 150 nL/min while spacing them with a stream of oil (Novec 7500 supplemented with 2% Krytox-Jeffamine 1000 diblock copolymer fluorosurfactant) infused into the chip (FIG. 31, inlet 4) at a flow-rate of 1200 nL/min. In parallel, RNA III-containing 100 pL droplets were reinjected (FIG. 31, inlet 1) at a frequency of ˜100 droplets per second by infusing the emulsion at 500 nL/min while spacing them with a stream of oil (Novec 7500 supplemented with 2% Krytox-Jeffamine 1000 diblock copolymer fluorosurfactant) infused into the chip (FIG. 31, inlet 2) at a flow-rate of 1300 nL/min. Pairs of droplets were allowed to form while the droplets were circulating into a short delay line. Pair-wised droplets were then fused when passing in front of an electrode pair to which a squared AC field (600 V, 30 Hz) was applied using a function generator connected to a high voltage amplifier (TREK Model 623B). At the same time, ˜15 pL of RT mixture (50 μL of reaction mixture were prepared by supplementing a CutSmart® buffer prepared at the recommended concentration with 1 mM dNTP, 20 μM coumarin acetate, 5 U of AMV reverse transcriptase (Life Science), and 0.01% Pluronic F68) was delivered to each droplet by infusing the mixture into the chip (FIG. 31, inlet 5) at 300 nL/min. Fused and picoinjected droplets were collected (FIG. 31, outlet 6) for 20 minutes in a 0.5 mL tube under mineral oil. Upon an incubation of 60 minutes at 42° C., ˜200 droplets were introduced in a tube and the emulsion was broken using of 1H, 1H, 2H, 2H, perfluoro-1-octanol (Sigma-Aldrich) in the presence of 0.2 mg/mL yeast total RNA (Ambion).

Illu1 adaptor was then added to cDNA by introducing 25 μL of aqueous phase into a mixture containing 1 μM of the RNAIII-Fw-89-Lg (molecule 37; displays the 19 nucleotides of Illu1 at its 5′ end) and 1 μM of the primer Illu-2-Fwd (molecule 6), 0.2 mM of each dNTP, 20 U of Q5 DNA polymerase (New England Biolabs) and the corresponding buffer at the recommended concentration. Upon 5 rounds of thermocycling (10 sec at 98° C., 30 sec at 60° C. and 3 min at 72° C.), 3 μL of amplification mixture were introduced in 120 μL of indexing mixture containing Q5 DNA polymerase (New England Biolabs) and its buffer at the recommended concentration, 0.2 mM of each dNTP and Nextera primers (here N704 and N517, Illumina) at the recommended concentration. Indexing was performed by thermocycling the mixture using the program recommended by the manufacturer. Indexing products were then purified using SeraMag beads as recommended (GE Healthcare) and the recovered products were analyzed on a bioanalyzer device (Agilent). Finally, the library was analyzed on MiSeq device using a V3-150 reagent kit (Illumina). A total of >20 millions reads for which the presence of both the barcodes and the RNA III-derived cDNA was confirmed were finally obtained.

Results

The quality control analysis on an Agilent device demonstrated that a single intense band was obtained (FIG. 32A) demonstrating the quality of the preparation process described in this example. Moreover, sequence analysis confirmed that vast majority (i.e. 99.75%) of the reads other than PhiX actually corresponded to RNA III-derived labelled cDNAs (FIG. 32B). Altogether, the data collected in this example demonstrated the capacity of the invention to successfully prepare prelabelled capture molecules (i.e. RT primer) in droplets prior to using them to reverse transcribe target RNA and by doing so to generate a barcoded cDNA compatible for indexing and sequencing. The presence of such barcode will then allow to cluster together RNAs originating from the same droplet (see example 2 section c and d).

### REFERENCES

Benito, Y., et al. RNA, 2000. 6(5): p. 668-79.
Brown, R. B. & Audet, J. R. Soc. Interface 5, S131-S138 (2008)
De Lange et al. Biomicrofluidics. 2016 Mar. 22; 10(2):024114
Islam et al. Nat. Protoc. 2012; 7:813-828.
Kintses et al. Chem. Biol. 2012. 19, 1001-1009
Klein et al., Cell. 2015. 161, 1187-1201
Macosko et al. Cell. 2015, 161, 1202-1214
Mazutis, L., et al., Analytical Chemistry, 2009. 81(12): p. 4813-21.
Novak et al. Angew. Chem. Int. Ed. 50, 390-395 (2011)
Rau et al. Appl. Phys. Lett. 2004. 84, 2940-2942
Rotem et al., PLoS ONE, 2015, 10(5): e0116328
Ryckelynck, M., et al., RNA, 2015. 21(3): p. 458-69.
Streets et al. Proc. Natl. Acad. Sci. USA, 2014, 111, 7048-7053
Xia, Y. N. and G. M. Whitesides, Soft lithography. Angewandte Chemie-International Edition, 1998. 37(5): p. 551-575.

1-18. (canceled)

19. A method of labelling a plurality of molecular targets from a plurality of entities while preserving the integrity of the single-entity information, said method comprising
providing a first set of emulsion droplets comprising droplets containing molecular targets, wherein each of these droplets comprises a plurality of molecular targets originating from no more than one entity;
providing a second set of emulsion droplets comprising droplets containing probes,
wherein each probe comprises a capture moiety capable of specific binding or ligation to a molecular target contained in droplets of the first set or to an adaptor linked to said molecular target, and a DNA moiety comprising an identification sequence,
wherein each identification sequence comprises a molecular identification (UMI) barcode, an entity identification (UEI) barcode and a calibrator (UEI-calibrator) barcode,
wherein each droplets of the second set comprises one or several UEI barcodes and one or several UEI-calibrator barcodes, the combination of UEI barcodes and UEI-calibrator barcodes being different for each droplet of the second set, and
wherein each identification sequence contained in a droplet of the second set comprises a UMI barcode which is different from the other identification sequences contained in the same droplet,
fusing droplets of the first set with droplets of the second set wherein a droplet of the first set is fused with no more than one droplet of the second set; and
labelling each molecular target with an identification sequence.

20. The method of claim 19, wherein the method further comprises encapsulating a plurality of entities within emulsion droplets, each droplet containing no more than one entity, and optionally lysing said entities within the droplets to release molecular targets,
thereby obtaining the first set of emulsion droplets.

21. The method of claim 19, wherein the method further comprises
encapsulating a plurality of entity identification (UEI) sequences, a plurality of calibrator (UEI-calibrator) sequences and a plurality of molecular identification (UMI) molecules with an amplification reaction mixture within emulsion droplets,
wherein each droplet comprising one or several UEI sequences, one or several UEI-calibrator sequences and a plurality of UMI molecules, the combination of UEI sequences and UEI-calibrator sequences being different for each droplet and each droplet comprising a plurality of UMI molecules,
wherein each UEI sequence comprises a UEI barcode and one or two overhang producing restriction sites,
each UEI-calibrator sequence comprises a UEI-calibrator barcode and one or two overhang producing restriction sites,
each UMI molecule comprises a capture moiety capable of specific binding or ligation to a molecular target or to an adaptor linked to said molecular target, and a DNA moiety comprising (i) a region proximal to the capture moiety and comprising a UMI barcode and (ii) a region distal from the capture moiety and comprising an overhang or an overhang producing restriction site, and
each UMI molecule comprises a UMI barcode which is different from the other UMI molecules contained in the same droplet;
amplifying UEI sequences and UEI-calibrator sequences within droplets; and
assembling UEI-calibrator barcodes, UEI barcodes and UMI molecules through restriction enzyme digestion and ligation of compatible overhangs,
thereby obtaining the second set of emulsion droplets.

22. The method of claim 21, wherein (a) UEI sequences and UEI-calibrator sequences are assembled through restriction enzyme digestion and ligation of compatible overhangs before amplification, and then (b) UMI molecules and amplification products are assembled through restriction enzyme digestion and ligation of compatible overhangs.

23. The method of claim 19, wherein at least some of molecular targets are nucleic acids and at least some probes comprise a capture moiety which is a single stranded DNA region which drives the specific recognition of a nucleic acid molecular target through conventional Watson-Crick base-pairing interactions.

24. The method of claim 23, wherein said nucleic acid molecular targets are labelled using said probes as priming sites for a DNA polymerase synthetizing complementary strands of molecular targets.

25. The method of claim 23, wherein at least some of molecular targets are RNA molecules and the DNA polymerase is a reverse transcriptase.

26. The method of claim 19, wherein at least some probes comprise a capture moiety which is
(i) a binding moiety that specifically binds to a molecular target and is directly bound to the DNA moiety,
(ii) a chimeric protein comprising a first domain that specifically binds to a molecular target and a second domain that binds to the DNA moiety, or
(iii) a binding moiety that binds specifically to a molecular target and a protein bridge, said protein bridge comprising a first domain that binds to the binding moiety and a second domain that binds to the DNA moiety.

27. The method of claim 26, wherein (i) the binding moiety or the first domain of the chimeric protein is selected from the group consisting of an antibody, a ligand of a ligand/anti-ligand couple, a peptide aptamer, a nucleic acid aptamer, a protein tag, or a chemical probe reacting specifically with a molecular target or a class of molecular targets, (ii) the first domain of the protein bridge is an immunoglobulin-binding bacterial protein, and/or (iii) the second domain of the protein bridge or the chimeric protein is selected from the group consisting of SNAP-tag, CLIP-tag or Halo-Tag.

28. The method of claim 19, wherein at least some probes comprise a capture moiety comprising an antibody moiety specific to a molecular target and a protein bridge, said protein bridge comprising a first domain that binds to a Fc region of the antibody moiety and a second domain that binds to the DNA moiety.

29. The method of claim 19, wherein at least one step of the method is implemented using a microfluidic system.

30. The method of claim 29, wherein a microfluidic system is used to generate the first set of emulsion droplets, and/or a microfluidic system is used to generate the second set of emulsion droplets, and/or a microfluidic system is used to fuse droplets of the first set with droplets of the second set.

31. The method of claim 29, wherein the method is implemented using a microfluidic comprising
a first emulsion re-injection module or on-chip droplet generation module;
a second emulsion re-injection module or on-chip droplet generation module;
a droplet-pairing module; and
optionally a module coupling droplet fusion to injection,
wherein emulsion re-injection modules and/or on-chip droplet generation modules are in fluid communication and upstream to the droplet-pairing module, the droplet-pairing module is in fluid communication and upstream to the module coupling droplet fusion to injection.

32. The method of claim 19, wherein the entity is a cell, a particle or an emulsion droplet.

33. The method of claim 32, wherein the emulsion droplet is an oil-in-water emulsion droplet exposing molecular targets on its outer surface.

34. A method of quantifying one or several molecular targets from a plurality of entities with single-entity resolution, said method comprising
labelling said molecular targets according to the method of claim 19;
capturing said labelled molecular targets;
amplifying sequences comprising UMI, UEI and UEI-calibrator barcodes;
sequencing amplified sequences.

35. A kit comprising:
a) a microfluidic device and/or
one or several probes; and/or
one or several UMI molecules; and/or
one or several UEI sequences; and/or
one or several UEI-calibrator sequences; and/or
one or several primers suitable to amplify UEI sequences and/or UEI-calibrator sequences, the probes, UMI molecules, UEI sequences, UEI-calibrator sequences being as defined in claim 19; and
optionally
an aqueous phase and/or an oil phase; and/or
a leaflet providing guidelines to use said a kit.

36. The kit of claim 35, wherein the microfluidic device comprises
a first emulsion re-injection module or on-chip droplet generation module;
a second emulsion re-injection module or on-chip droplet generation module;
a droplet-pairing module; and
optionally a module coupling droplet fusion to injection,
wherein emulsion re-injection modules and/or on-chip droplet generation modules are in fluid communication and upstream to the droplet-pairing module, the droplet-pairing module is in fluid communication and upstream to the module coupling droplet fusion to injection.
